(19) United States
(12) Patent Application Publication
(10) Pub. No.: US 2002/0187522 A1
(43) Pub. Date: Dec. 12, 2002

(54) DNA encoding human Cid1

(76) Inventors: Yan Luo, Lake Bluff, IL (US); Vincent L. Giranda, Gurnee, IL (US)

Correspondence Address: Steven F. Weinstock, Abbott Laboratories, Department 377 / AP6D-2, 100 Abbott Park Road, Abbott Park, IL, 60064-6050, US

(21) Appl. No.: 09/800,362

(22) Filed: Mar. 6, 2001

(51) Int. Cl. C12Q 1/68; G01N 33/53; C07H 21/04; A61K 48/00; C07K 14/705

(52) U.S. Cl. 435/69.1; 435/6; 435/7.1; 435/320.1; 435/325; 530/350; 514/44; 536/23.5

(57) ABSTRACT

An isolated and purified nucleic acid sequence encoding the human Cid1 gene and the polypeptide it encodes is disclosed.

### BACKGROUND OF THE INVENTION

Genome integrity is maintained by a complex network of checkpoint mechanisms that coordinate DNA replication with repair and ensure the correct ordering of cell cycle events (Hartwell and Weinert, Science, 246, 629 (1989)). These checkpoints can be disrupted by a variety of drugs or genetic lesions. In mammalian cells, loss of checkpoint control results in DNA rearrangements, amplification and chromosome loss, events that are causally associated with cancer (Hartwell and Kastan, Science, 266, 1821 (1994); Lehmann and Carr, Trends Genet., 11, 375 (1995)).

In fission and budding yeasts, relief-of-dependence mutations have been identified that allow cell cycle progression under conditions that would normally cause cell cycle arrest (reviewed by Murray, Nature, 359, 599 (1992)). Genetic analysis of these mutants has provided important information about the mechanisms of checkpoint control, and from these studies a picture of how checkpoints may work at the molecular level is beginning to emerge.

Two of the most extensively characterized pathways are the S-M checkpoint, which prevents cells from entering mitosis with incompletely replicated chromosomes, and the DNA damage checkpoint, which prevents entry into mitosis (or anaphase) when DNA integrity is compromised (Stewart and Enoch, Curr. Op. Cell Biol., 8, 781 (1996)). Genetic evidence has indicated that the DNA damage and S-M checkpoint pathways are distinct in the fission yeast Schizosaccharomyces pombe (Enoch and Nurse, Cell, 60, 665 (1990)), although several rad/hus mutants that are defective in DNA damage checkpoints also show sensitivity to the DNA replication inhibitor hydroxyurea (HU) (Al-Khodairy and Carr, EMBO J., 11, 1343 (1992);

Enoch et al., Genes Dev., 6, 2035 (1992)). This suggests that there is a degree of overlap between the S-M and DNA damage checkpoints in terms of the gene products involved.

Several chemical agents have been identified that can override checkpoint control, including the phosphatase inhibitors okadaic acid (Yamashita et al., EMBO J., 13, 4331 (1990)), fostriecin (Roberge et al., Cancer Res., 54, 6115 (1994); Guo et al, EMBO J., 14, 976 (1995)) and calyculin A (Nakamura et al., Cancer Res., 54, 2088 (1994)), protein kinase antagonists, such as aminopurines (Andreassen et al., PNAS, 89, 2272 (1992)), and methylxanthines such as pentoxifylline and caffeine. Caffeine has been known to override the S/M checkpoint in animal cells (Schlegel et al. Science, 232, 1264 (1986)). In addition, caffeine also overrides the G2/M checkpoint in mammalian cells. When cells are subjected to DNA replication block induced by HU in the presence of caffeine, Chkl activation is inhibited and (Akiko Kumagai, Zijian Guo, Katayoon H. Emami, Sophiex Wang and William G. Dunphy. The Xenopus Chkl Protein Kinase Meditates a Caffeine-sensitive Pathway of Checkpoint Control in cell free extracts. The J. of Cell Biology 142 (f): 1559-1569. The cells will progress into mitosis prematurely (catastrophic mitosis) and subsequently die. Caffeine also induces cell death directly from S phase arrest. The abrogation of cell cycle arrest by caffeine is associated with the selective sensitization of p53-deficient primary and tumor cells to anticancer agents (Russell et al., Cancer Res., 55, 1639 (1995); Powell et al., Cancer Res., 55, 1643 (1995); Fan et al., Cancer Res., 55, 1649 (1995); Yao et al., Nature Med., 2, 1140 (1996)).

Caffeine also disrupts the S/M checkpoint in the fission yeast S. pombe. In the presence of HU, the caffeine-induced disruption results in cell death. However, in S. pombe the lethality of a combination of caffeine with the DNA replication inhibitor HU is suppressed by overexpression of either of two S. pombe genes, Cid1 or Cid2 (Wang et al., Journal of Cell Science, 112, 927 (1999)). Cid1 belongs to a divergent protein family that includes Trf4 and Trf5 in Saccharomyces cerevisiae. Cells lacking Cid 1 are viable except they are sensitive to the combination treatment with Hydroxyurea and caffeine. In addition, cells lacking Cid 1 are also defective in S-M checkpoint mechanism in the absence of Cds 1. (S-W. Wang, T. Toda, R. Nacallum, A. L. Harris, and C. Norbury. Cid 1. A Fission Yeast Protein Required for S-M Ceckpoint Control. Mol. Cel. Biol. 20 (9):3234-3244). However, the human homologs of Cid1 and Cid2, which may play a role in DNA damage repair, have not been identified.

Thus, what is needed is the identification and isolation of the human homologs of Cid1 and Cid2.

### SUMMARY OF THE INVENTION

The invention provides an isolated and purified nucleic acid molecule comprising a nucleic acid sequence that is structurally related to Schizosaccharomyces pombe nucleic acid encoding Cid1 (SEQ ID NO:7 encoded by SEQ ID NO:8) and encodes a human polypeptide, a biologically active portion (fragment) thereof, or the complement thereof As described herein, the S. pombe Cid1 gene was employed to identify structurally related human DNAs in a database. This resulted in the identification of three different open reading frames, Cid1-hu4 (SEQ ID NO:1), Cid1-hu6 (SEQ ID NO:3), and Cid1-hu11 (SEQ ID NO:5). As the DNA damage response is conserved throughout the eukaryotic kingdom, the human Cid1 genes likely 5 function in response to DNA damage in human cells. As used herein, a “biologically active portion or fragment” of a nucleic acid molecule of the invention is one which is greater than 7 nucleotides in length and hybridizes under moderate, or more preferably stringent, conditions to SEQ ID NO: 1, SEQ ID NO:3, SEQ ID NO:5, or the complement thereof Moderate and stringent hybridization conditions are well known to the art, see, for example sections 9.47-9.51 of Sambrook et al. (Molecular Cloning: A Laboratory Manual, Cold Spring Harbor Laboratory, Cold Spring Harbor, N.Y. (1989), which are incorporated by reference herein. For example, stringent conditions are those that (1) employ low ionic strength and high temperature for washing, for example, 0.015 M NaCl/0.0015 M sodium citrate (SSC); 0.1% sodium lauryl sulfate (SDS) at 50° C., or (2) employ a denaturing agent such as formamide during hybridization, e.g., 50% formamide with 0. 1% bovine serum albumin/0.1% Ficoll/0.1% polyvinylpyrrolidone/50 mM sodium phosphate buffer at pH 6.5 with 750 mM NaCI, 75 mM sodium citrate at 42° C. Another example is use of 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% sodium dodecylsulfate (SDS), and 10% dextran sulfate at 42° C., with washes at 42° C. in 0.2×SSC and 0.1% SDS. Preferred nucleic acid molecules of the invention comprise a nucleic acid sequence which encodes a polypeptide comprising SEQ ID NO:2 (e.g., encoded by SEQ ID NO: 1), SEQ ID NO:4 (e.g., encoded by SEQ ID NO:3), and SEQ ID NO:6 (e.g., encoded by SEQ ID NO:5).

“Structurally related” nucleic acid molecules, as used herein, includes nucleic acid molecules which are identified using parameters such as those described in Example I, nucleic acid molecules having at least 80% nucleic acid sequence identity to SEQ ID NO: 1, SEQ ID NO:3, SEQ ID NO:5, or the complement thereof, and nucleic acid molecules which hybridize under moderate, more preferably stringent, hybridization conditions to SEQ ID NO: 1, SEQ ID NO:3, SEQ ID NO: 5, or the complement thereof The percent identity two sequences, whether nucleic acid or peptide sequences divided by length of the shorter sequences and multiplied by 100. An approximate alignment for nucleic acid sequences is provided by the local homology algorithm of Smith and Waterman, Advances in Applied Mathematics 2:482-489 (1981). This algorithm can be extended to use with peptide sequences using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An implementation of this algorthium for nucleic and peptide sequences is provided by the Genetics Computer Group (Madison, Wis.) in thier Best Fit utility application. The default parameters for this method are described in the Wisconsin Sequence Analysis Package Program Manual, Version 8 (1995) (available from Genetics Computer Group, Madison, Wis.). Other equally suitable programs for calculating the percent identity or similarity between sequences are generally known in the art. Thus, the nucleic acid molecules of the invention include variant nucleic acid molecules which have nucleotide base substitutions, insertions and/or deletions relative to SEQ ID NO: 1, SEQ ID NO:3 or SEQ ID NO:5. The nucleic acid molecules of the invention may comprise RNA, DNA, e.g., cDNA or genomic DNA, or a combination thereof, and are useful in methods to detect expression of human Cid₁ genes.

The invention also provides an expression cassette comprising a promoter functional in a host cell operably linked to an isolated and purified nucleic acid molecule comprising a nucleic acid sequence that is structurally related to Schizosaccharomyces pombe nucleic acid encoding Cid1 and encodes a human polypeptide, a biologically active portion thereof, or the complement thereof The promoter may be functional in a prokaryotic cell, e.g., E. coli, or a eukaryotic cell, e.g., a yeast or a mammalian cell, including, but not limited to, human, hamster, murine, ovine, canine, bovine, equine, caprine, and feline cells. The expression cassettes of the invention are useful to express the encoded polypeptide or antisense Cid1 sequences. The antisense expression cassettes of the invention preferably comprise nucleic acid molecules which are the exact complement of SEQ ID NO: 1, SEQ ID NO:3, SEQ ID NO:5, or a biologically active portion thereof With reference to antisense sequences, a “biologically active portion” means that the portion inhibits the expression of human Cid1, e.g., endogenous (native) human Cid1 in human cells, or recombinant human Cid1 in a transfected cell.

Hence, the invention further provides an isolated and purified polypeptide encoded by the nucleic acid molecule of the invention, or a biologically active portion thereof. For example, preferred isolated and purified polypeptides of the invention include a polypeptide comprising SEQ ID NO:2, SEQ ID NO:4, SEQ ID NO:6, a biologically active portion (fragment) thereof, as well as variants thereof As used herein, a “biologically active portion” of a polypeptide of the invention includes a peptide of at least seven amino acid residues that binds an antibody which specifically recognizes a polypeptide having SEQ ID NO.2, SEQ ID NO;4, SEQ ID NO:6, or a variant thereof A variant of SEQ ID NO:2, SEQ ID NO:4, or SEQ ID NO:6 is a polypeptide that has at least about 80%, preferably at least about 90%, but less than 100%, contiguous amino acid sequence homology or identity to the amino acid sequence corresponding to SEQ ID NO:2, SEQ ID NO:4, or SEQ ID NO:6. Thus, a variant Cid1 polypeptide of the invention may include amino acid residues not present in SEQ ID NO:2, SEQ ID NO:4, or SEQ ID NO:6, e.g., amino acid substitutions, and amino and/or carboxy, or internal, deletions or insertions, of amino acid residues relative to SEQ ID NO:2, SEQ ID NO:4, or SEQ ID NO:6. Variant polypeptides of the invention can include polypeptides having at least one D-amino acid, as well as moieties other than the amino acid residues that correspond to SEQ ID NO:2, SEQ ID NO:4, or SEQ ID NO:6, such as amino acid residues that form a part of a fusion protein, nucleic acid molecules or targeting moieties such as antibodies or fragments thereof Preferred variant polypeptides of the invention are those having conservative substitutions at positions that contain unconserved amino acids because they are more likely to tolerate changes.

The expression cassettes of the invention may be employed in a method of using the nucleic acid molecule of the invention to alter the amount of a human Cid 1 polypeptide in a cell. The method comprises transfecting a host cell with an isolated and purified nucleic acid molecule of the invention, or a biologically active portion thereof The nucleic acid sequence is expressed in the transfected host cell in an amount that alters the amount of the human Cid1 polypeptide produced by the transfected cell relative to the amount of human Cid1 in a corresponding untransfected cell. If the nucleic acid sequence is operatively linked to a promoter in the sense orientation, the amount of the recombinant polypeptide produced by the transfected host cell is increased relative to the amount of the polypeptide produced by the corresponding untransfected host cell. If the nucleic acid sequence is in an antisense orientation relative to a promoter, the amount of the polypeptide produced by the transfected host cell is decreased relative to the amount of the polypeptide produced by the corresponding untransfected host cell.

The invention further provides a method to produce a human Cid1 polypeptide, comprising: culturing a host cell transfected with a nucleic acid molecule comprising a nucleic acid sequence encoding a human Cid1 so that said host cell expresses the polypeptide or a biologically active portion thereof Preferably, the polypeptide is isolated from the host cell and purified. Therefore, the invention also provides isolated, purified human Cid1 polypeptide, or a biologically active portion thereof The polypeptides of the invention are useful to prepare antibodies, which in turn are useful to detect the polypeptide of the invention, e.g., in biological samples such as a physiological sample from a mammal. Physiological samples include fluid samples and tissue samples. The nucleic acid molecules, polypeptides and antibodies of the invention, e.g., in the form of a kit, may be useful in diagnostic as well as therapeutic applications.

Also provided is a method to detect human Cid1 nucleic acid. The method comprises contacting a nucleic acid sample from a human with an amount of at least one oligonucleotide under conditions effective to amplify human Cidi nucleic acid. Then the amplified nucleic acid is detected or determined. Alternatively, a nucleic acid sample from a human is contacted with a probe comprising at least a portion of human Cid1 nucleic acid in an amount and under conditions effective to form a binary complex between the Cid1 nucleic acid in the sample and the probe. Then the amount of complex formation is detected or determined.

The invention also provides an isolated and purified antibody which specifically binds to or recognizes a polypeptide of the invention, or a portion thereof Thus, the isolated polypeptide of the invention is useful in an immunogenic composition, preferably in combination with a pharmaceutically acceptable carrier, which, when administered to an animal, induces the production of antibodies to the polypeptide. Antibodies within the scope of the invention include monoclonal antibodies and polyclonal antibodies. Also provided is a hybridoma cell line which produces a monoclonal antibody of the invention.

Further provided is a method to detect a human Cid 1 polypeptide. The method comprises contacting a biological sample from a human with an antibody of the invention so as to form a binary complex. Complex formation is then detected or determined. The biological sample may comprise intact cells, or comprise a population of polypeptides isolated from a cellular source or prepared in vitro.

One of the challenges in cancer therapy is to decrease the toxicity of conventional cytotoxic agents. In particular, tumor cells are often defective in the DNA damage response, especially in the GI checkpoint. This renders tumor cells more dependent on the S/M and G2/M checkpoint mechanisms for survival and DNA repair, likely accounting for the increased sensitivity of tumor cells to chemotherapeutic agents or radiation relative to normal tissue.

Overexpressing Cid1 & Cid2 in S. Pombe rescues the lethality induced by HU and caffeine combination treatment. Human Cid 1 and Cid 2 are likely to function similarly. Since tumor cells are often defective in G1 checkpoint, inhibition of human Cid 1 may enhance the defects in tumor cells and cause specific killing of tumor cells. Thus, inhibitors of human Cid1 may be useful sensitize tumor cells to chemotherapy/radiotherapy. Hence, human Cid genes and their products are useful for the development of assays related to the screening of inhibitors of the gene products and cell cycle regulation. Thus, the invention provides a method to screen for an agent which inhibits the activity of a human Cid1 polypeptide. The method comprises treating a host cell which comprises DNA encoding the human polypeptide with the agent; and determining or detecting whether the agent inhibits the activity of the human polypeptide. For example, the cells may be tumor cells. The DNA encoding the human Cid1 polypeptide may be the native (endogenous) DNA or a recombinant DNA.

The invention also provides a method to identify an agent that is an inhibitor of a human Cid1 polypeptide, comprising: contacting an isolated and purified polypeptide of the invention with the agent; and detecting or determining whether the agent binds to or inhibits the activity of the polypeptide. An assay to determine delay of mitosis after DNA damages is discussed in Fumari, B., et al., (1997). A method to detect Cdc25 mitotic inducer targeted by chkl DNA damage checkpoint kinase is shown in Science 277: 1495-7. Methods to determine mitotic index after DNA damage can be found in Yu, L., Orlandi, L., Wang, P., Orr, M. S., Senderowicz, A. M., Sausville, E. A., Silvestrini, R., Watanabe, N., Piwnica-Worms, H., and O'Connor, P. M. (1998), UCN-01 Abrogates G2 arrest through a Cdc2-Dependent Pathway that is Associated with Inactivation of the Wee 1 Hu Kinase and Activation of the Cdc25C Phosphate, Journal of Biological Chemistry 273:33455-64.

### BRIEF DESCRIPTION OF THE DRAWINGS

FIG. 1 is a diagram illustrating the position of partial sequences of clones from different libraries which together span the entire length of the Cid1-hu4 cDNA.

FIG. 2 is the DNA sequence (SEQ ID NO: 1) and the predicted amino acid sequence (SEQ ID NO:2) of Cid1-hu4.

FIG. 3 illustrates a comparison between the predicted amino acid sequence of Cid1-hu4 (SEQ ID NO:2) and the amino acid sequence of a portion of Schizosaccharomyces pombe Cid1 (SEQ ID NO:7). Lines indicate identical residues, and a colon and dot indicates different levels of conserved change.

FIG. 4 is a diagram illustrating the position of partial sequences of clones from different libraries which together span the entire length of the Cid1-hu6 cDNA.

FIG. 5 is the DNA sequence (SEQ ID NO:3) and the predicted amino acid sequence (SEQ ID NO:4) of Cid1-hu6.

FIG. 6 illustrates a comparison between the predicated amino acid sequence of Cid1-hu6 (SEQ ID NO:4) and the amino acid sequence of a portion of Schizosaccharomyces pombe Cid1 (SEQ ID NO: 8).

FIG. 7 is a diagram illustrating the position of partial sequences of clones from different libraries which together span the entire length of the Cid1 -hu 11.

FIG. 8 is the DNA sequence (SEQ ID NO:5) and the predicted amino acid sequence (SEQ ID NO:6) for Cid1-hu11.

FIG. 9 illustrates a comparison between the predicated amino acid sequence of Cid1-hu11 (SEQ ID NO:6) and the amino acid sequence of Schizosaccharomyces pombe Cid.

FIG. 10 illustrates an alignment of Cid-1hu4, Cid1-hu6, and Cid 1-hu11.

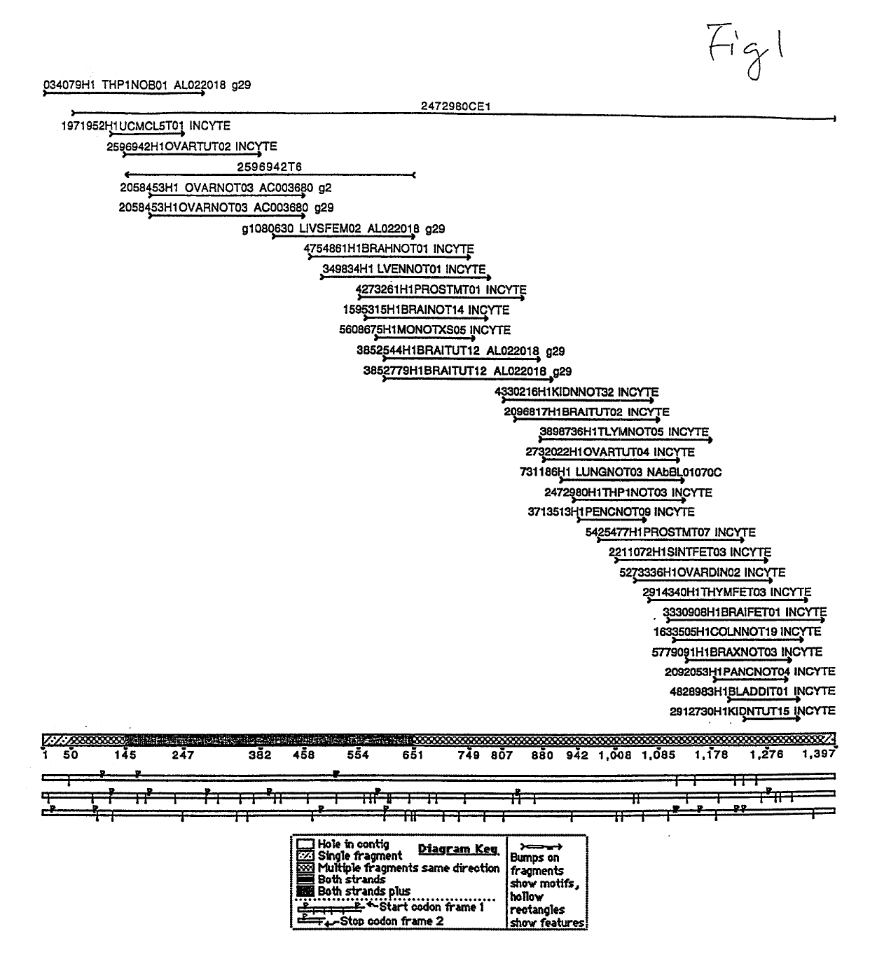

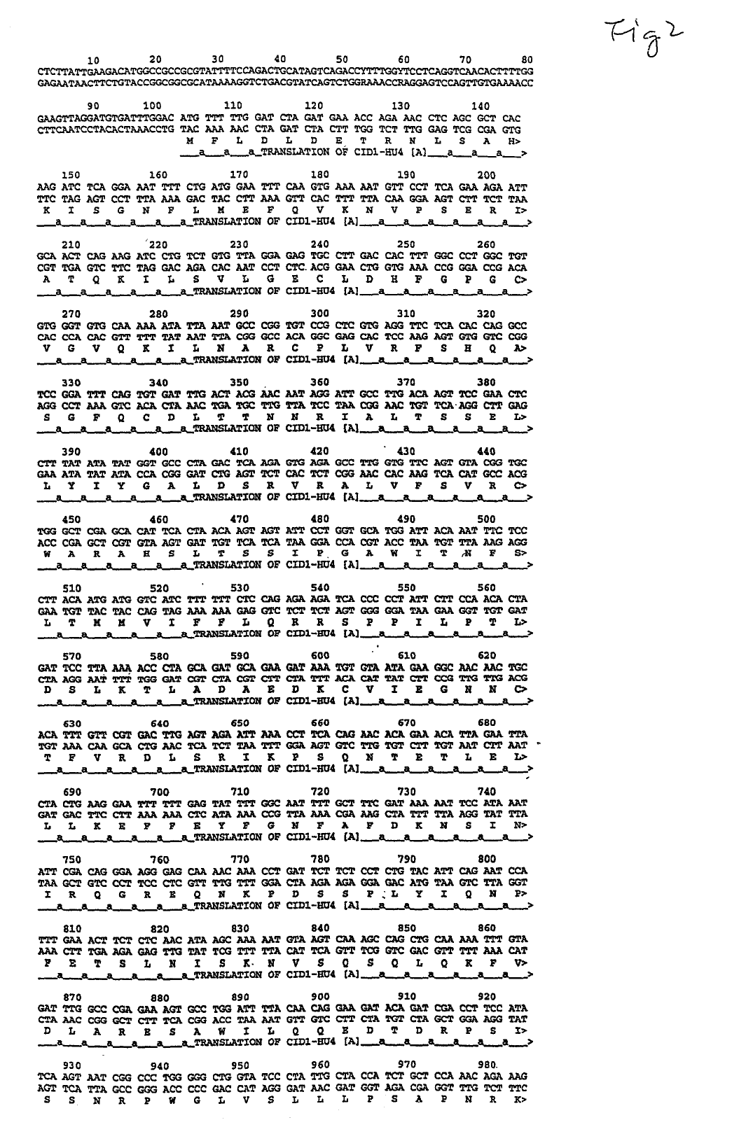

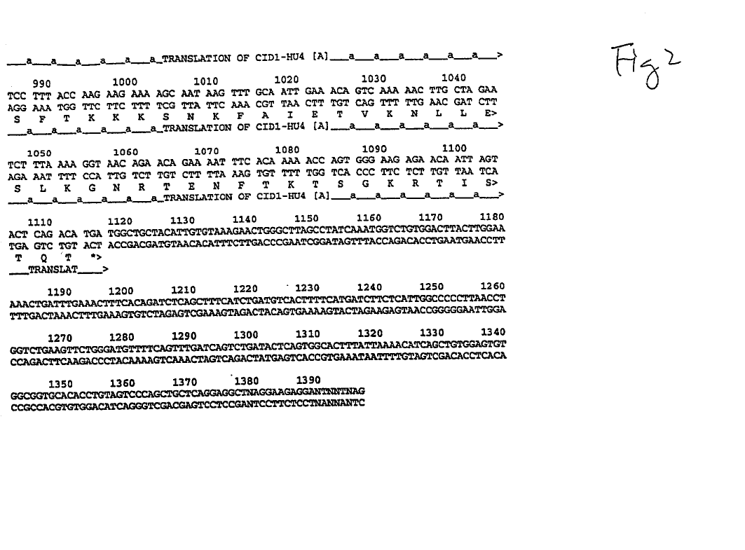

### DETAILED DESCRIPTION OF THE INVENTION

Definitions

As used herein, the terms “isolated and/or purified” refer to in vitro preparation, isolation and/or purification of a nucleic acid molecule or polypeptide of the invention, so that it is not associated with in vivo substances. Thus, with respect to an “isolated and purified nucleic acid molecule” encoding human Cid1 polypeptide, which includes either ribonucleotides or deoxynucleotides or a modified form of either type of nucleotide, including genomic DNA, cDNA, RNA, both naturally occuring as well as forms that are synthetic in origin, or some combination thereof, the “isolated and purified nucleic acid molecule” (1) is not associated with all or a portion of a polynucleotide in which the “isolated and purified nucleic acid molecule” is found in nature, (2) is operably linked to a polynucleotide which it is not linked to in nature (i.e., it is chimeric), or (3) does not occur in nature as part of a larger sequence. Thus, an isolated DNA is isolated from its natural cellular environment and components of the cells, such as nucleic acid or polypeptide, so that it can be sequenced, replicated, and/or expressed. The term includes single and double stranded forms of nucleic acid. For example, “isolated human Cid1 nucleic acid” is RNA or DNA containing greater than 200-500, preferably 500, and more preferably 600 or more, sequential nucleotide bases that encode at least a portion of human Cid1, or a RNA or DNA complementary thereto, or hybridizes, respectively, to RNA or DNA encoding Cid1 or the complement thereof, and remains stably bound under stringent conditions, as defined by methods well known in the art, e.g., in Sambrook et al. (Molecular Cloning: A Laboratory Manual, Cold Spring Harbor Laboratory, N.Y. (1989)). Thus, the RNA or DNA is “isolated” in that it is free from at least one contaminating nucleic acid with which it is normally associated in the natural source of the RNA or DNA and is preferably substantially free of any other RNA or DNA. The phrase “free from at least one contaminating source nucleic acid with which it is normally associated” includes the case where the nucleic acid is reintroduced into the source or natural cell but is in a different chromosomal location or is otherwise flanked by nucleic acid sequences not normally found in the source cell.

The term “oligonucleotide” or “primer” referred to herein includes naturally occurring, and modified nucleotides linked together by naturally occurring, and non-naturally occurring oligonucleotide linkages. Oligonucleotides are a polynucleotide subset with 200 bases or fewer in length. Preferably, oligonucleotides are 10 to 60 bases in length and most preferably 12, 13, 14, 15, 16, 17, 18, 19, or 20 to 40 bases in length. Oligonucleotides are usually single stranded, e.g., for probes, although oligonucleotides may be double stranded, e.g, for use in the construction of a variant. Oligonucleotides can be either sense or antisense oligonucleotides. The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotide linkages such as phosphorothioate, phosphorodithioate, phosphoroselenoate, phosphorodeselenoate, phosphoranilothioate, phosphoraniladate, phosphoroamidate, and the like. An oligonucleotide can include a label for detection, if desired.

The term “isolated polypeptide” means a polypeptide encoded by genomic DNA, cDNA or recombinant RNA, or is of synthetic origin, or some combination thereof, which isolated polypeptide (1) is not associated with proteins found in nature, (2) is free of other proteins from the same source, e.g., free of human proteins, (3) is expressed by a cell from a different species, or (4) does not occur in nature.

The term “sequence homology” means the proportion of base matches between two nucleic acid sequences or the proportion amino acid matches between two amino acid sequences. When sequence homology is expressed as a percentage, e.g., 50%, the percentage denotes the proportion of matches over the length of sequence from, e.g. a sequence encoding human Cid1, that is compared to some other sequence. Gaps (in either of the two sequences) are permitted to maximize matching; gap lengths of 15 bases or less are usually used, 6 bases or less are preferred with 2 bases or less more preferred. When using oligonucleotides as probes or treatments, the sequence homology between the target nucleic acid and the oligonucleotide sequence is generally not less than 17 target base matches out of 20 possible oligonucleotide base pair matches (85%); preferably not less than 9 matches out of 10 possible base pair matches, and more preferably not less than 19 matches out of 20 possible base pair matches (95%).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, 1972, volume 5, National Biomedical Research Foundation, pp. 101-110, and Supplement 2 to this volume, pp. 1-10. The two sequences or parts thereof are more preferably homologous if their amino acids are greater than or equal to 50% identical when optimally aligned using the ALIGN program.

The following terms are used to describe the sequence relationships between two or more polynucleotides: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing, or may comprise a complete cDNA or gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity.

A “comparison window”, as used herein, refers to a conceptual segment of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (1981) Adv. Appl. Math 2: 482, by the homology alignment algorithm of Needleman and Wunsch (1970) J. Mol. Biol. 48: 443, by the search for similarity method of Pearson and Lipman (1988) Proc. Natl. Acad. Sci. (U.S.A.) 85: 2444, by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence 10 identity. The terms “substantial identity” as used herein denote a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

As applied to polypeptides, the term “substantial identity” means that two peptide sequences, when optimally aligned, such as by the programs GAP or BESTFIT using default gap 20 weights, share at least about 80 percent sequence identity, preferably at least about 90 percent sequence identity, more preferably at least about 95 percent sequence identity, and most preferably at least about 99 percent sequence identity.

As used herein, the term “recombinant” e.g., “recombinant Cid1 gene” refers to a nucleic acid, e.g., to DNA, that has been derived or isolated from any appropriate tissue source, that may be subsequently chemically altered in vitro, so that its sequence is not naturally occurring, or corresponds to naturally occurring sequences that are not positioned as they would be positioned in a genome which has not been transformed with exogenous DNA. An example of a DNA “derived” from a source, would be a DNA sequence that is identified as a useful fragment within a given organism, and which is then chemically synthesized in essentially pure form. An example of such DNA “isolated” from a source would be a useful DNA sequence that is excised or removed from said source by chemical means, e.g., by the use of restriction endonucleases, so that it can be further manipulated, e.g., amplified, for use in the invention, by the methodology of genetic engineering.

Thus, recovery or isolation of a given fragment of DNA from a restriction digest can employ separation of the digest on polyacrylamide or agarose gel by electrophoresis, identification of the fragment of interest by comparison of its mobility versus that of marker DNA fragments of known molecular weight, removal of the gel section containing the desired fragment, and separation of the gel from DNA. See Lawn et al., Nucleic Acids Res., 9, 6103 (1981), and Goeddel et al., Nucleic Acids Res., 8, 4057 (1980). Therefore, a DNA of the invention includes completely synthetic DNA sequences, semi-synthetic DNA sequences, DNA sequences isolated from biological sources, and DNA sequences derived from RNA, as well as mixtures thereof As used herein, the term “derived” with respect to a RNA molecule means that the RNA molecule has complementary sequence identity to a particular DNA molecule.

“Host cell” means a cell into which a DNA or RNA molecule of the invention is delivered. A “host cell” may be any cell, including prokaryotic and eukaryotic cells, e.g., mammalian cells such as human cells.

I. Nucleic Acid Molecules of the Invention

A. Sources of the Nucleic Acid Molecules of the Invention

Sources of nucleotide sequences from which the present nucleic acid molecules encoding at least a portion of a human Cid 1 polypeptide, or the nucleic acid complement thereof, include total or polyA⁺RNA from any human cellular source from which cDNAs can be derived by methods known in the art. Other sources of the DNA molecules of the invention include genomic libraries derived from any human cellular source.

B. Isolation of the Nucleic Acid Molecules of the Invention

A nucleic acid molecule encoding a human Cid1 polypeptide or peptide can be identified and isolated using standard methods, as described by Sambrook et al., Molecular Cloning: A Laboratory Manual, Cold Spring Harbor, N.Y. (1989). For example, reverse-transcriptase PCR (RT-PCR) can be employed to isolate and clone Cid1 DNA. Oligo-dT can be employed as a primer in a reverse transcriptase reaction to prepare first-strand cDNAs from isolated RNA which contains RNA sequences of interest. RNA can be isolated by methods known to the art, e.g., using TRIZOL™ reagent (GIBCO-BRL/Life Technologies, Gaithersburg, Md.). Resultant first-strand cDNAs are then amplified in PCR reactions.

“Polymerase chain reaction” or “PCR” refers to a procedure or technique in which amounts of a preselected fragment of nucleic acid, RNA and/or DNA, are amplified as described in U.S. Pat. No. 4,683,195. Generally, sequence information from the ends of the region of interest or beyond is employed to design oligonucleotide primers comprising at least 7-8 nucleotides. These primers will be identical or similar in sequence to opposite strands of the template to be amplified. PCR can be used to amplify specific RNA sequences, specific DNA sequences from total genomic DNA, and cDNA transcribed from total cellular RNA, bacteriophage or plasmid sequences, and the like. See generally Mullis et al., Cold Spring Harbor Symp. Quant. Biol., 51, 263 (1987); Erlich, ed., PCR Technology, (Stockton Press, New York, 1989). Thus, PCR-based cloning approaches rely upon conserved sequences deduced from alignments of related gene or polypeptide sequences.

Primers are made to correspond to highly conserved regions of polypeptides or nucleotide sequences which were identified and compared to generate the primers, e.g., by a sequence comparison of isolated Cid1 genes. One primer is prepared which is predicted to anneal to the antisense strand, and another primer prepared which is predicted to anneal to the sense strand, of a DNA molecule which encodes a Cid1 polypeptide.

The products of each PCR reaction are separated via an agarose gel and all consistently amplified products are gel-purified and cloned directly into a suitable vector, such as a known plasmid vector. The resultant plasmids are subjected to restriction endonuclease and dideoxy sequencing of double-stranded plasmid DNAs.

Another approach to identify, isolate and clone cDNAs which encode human Cid1, is to screen a cDNA library. Screening for DNA fragments that encode all or a portion of a cDNA encoding human Cid1 can be accomplished by probing the library with a probe which has sequences that are highly conserved between genes believed to be related to Cid1, e.g., the homolog of a human Cid1 from a different species, or by screening of plaques for binding to antibodies that specifically recognize Cid1. DNA fragments that bind to a probe having sequences which are related to Cid1, or which are immunoreactive with antibodies to Cid1, can be subcloned into a suitable vector and sequenced and/or used as probes to identify other cDNAs encoding all or a portion of Cid1.

C. Variants of the Nucleic Acid Molecules of the Invention

Nucleic acid molecules encoding amino acid sequence variants of a Cid1 polypeptide or peptide are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of, for example, Cid1.

Oligonucleotide-mediated mutagenesis is a preferred method for preparing amino acid substitution variants of a peptide or polypeptide. This technique is well known in the art as described by Adelman et al., DNA, 2, 183 (1983). Briefly, DNA is altered by hybridizing an oligonucleotide encoding the desired mutation to a DNA template, where the template is the single-stranded form of a plasmid or bacteriophage containing the unaltered or native DNA sequence of Cid1, or a portion thereof After hybridization, a DNA polymerase is used to synthesize an entire second complementary strand of the template that will thus incorporate the oligonucleotide primer, and will code for the selected alteration in Cid1.

Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridize properly to the single-stranded DNA template molecule. The oligonucleotides are readily synthesized using techniques known in the art such as that described by Crea et al., Proc. Natl. Acad. Sci. U.S.A., 75, 5765 (1978).

The DNA template can be generated by those vectors that are either derived from bacteriophage M13 vectors (the commercially available M13mp18 and M13mp19 vectors are suitable), or those vectors that contain a single-stranded phage origin of replication as described by Viera et al., Meth. Enzymol., 153, 3 (1987). Thus, the DNA that is to be mutated may be inserted into one of these vectors to generate single-stranded template. Production of the single-stranded template is described in Sections 4.21-4.41 of Sambrook et al., Molecular Cloning: A Laboratory Manual (Cold Spring Harbor Laboratory Press, N.Y. 1989).

Alternatively, single-stranded DNA template may be generated by denaturing double-stranded plasmid (or other) DNA using standard techniques.

For alteration of the native DNA sequence (to generate anino acid sequence variants, for example), the oligonucleotide is hybridized to the single-stranded template under suitable hybridization conditions. A DNA polymerizing enzyme, usually the Klenow fragment of DNA polymerase I, is then added to synthesize the complementary strand of the template using the oligonucleotide as a primer for synthesis. A heteroduplex molecule is thus formed such that one strand of DNA encodes the mutated form of Cid1, and the other strand (the original template) encodes the native, unaltered sequence of Cid1. This heteroduplex molecule is then transformed into a suitable host cell, usually a prokaryote such as E. Coli JM101. After the cells are grown, they are plated onto agarose plates and screened using the oligonucleotide primer radiolabeled with 32-phosphate or 33-phosphate to identify the bacterial colonies that contain the mutated DNA. The mutated region is then removed and placed in an appropriate vector for peptide or polypeptide production, generally an expression vector of the type typically employed for transformation of an appropriate host.

The method described immediately above may be modified such that a homoduplex molecule is created wherein both strands of the plasmid contain the mutations(s). The modifications are as follows: The single-stranded oligonucleotide is annealed to the single-stranded template as described above. A mixture of three deoxyribonucleotides, deoxyriboadenosine (dATP), deoxyriboguanosine (dGTP), and deoxyribothymidine (dTTP), is combined with a modified thiodeoxyribocytosine called dCTP-(S) (which can be obtained from the Amersham Corporation). This mixture is added to the template-oligonucleotide complex. Upon addition of DNA polymerase to this mixture, a strand of DNA identical to the template except for the mutated bases is generated. In addition, this new strand of DNA will contain dCTP-(S) instead of dCTP, which serves to protect it from restriction endonuclease digestion.

After the template strand of the double-stranded heteroduplex is nicked with an appropriate restriction enzyme, the template strand can be digested with ExoIII nuclease or another appropriate nuclease past the region that contains the site(s) to be mutagenized. The reaction is then stopped to leave a molecule that is only partially single-stranded. A complete double-stranded DNA homoduplex is then formed using DNA polymerase in the presence of all four deoxyribonucleotide triphosphates, ATP, and DNA ligase. This homoduplex molecule can then be transformed into a suitable host cell such as E. Coli JM 101.

II. Preparation of Agents Falling Within the Scope of the Invention

A. Nucleic Acid Molecules

1. Chimeric Expression Cassettes

To prepare expression cassettes for transformation herein, the recombinant or selected DNA sequence or segment may be circular or linear, double-stranded or single-stranded. A DNA sequence which encodes an RNA sequence that is substantially complementary to a mRNA sequence encoding a Cid1 polypeptide is typically a “sense” DNA sequence cloned into a cassette in the opposite orientation (i.e., 3 to 5 rather than 5 to 3 ). Generally, the DNA sequence or segment is in the form of chimeric DNA, such as plasmid DNA, that can also contain coding regions flanked by control sequences which promote the expression of the selected DNA present in the resultant cell line.

As used herein, “chimeric” means that a vector comprises DNA from at least two different species, or comprises DNA from the same species, which is linked or associated in a manner which does not occur in the “native” or wild type of the species.

Aside from DNA sequences that serve as transcription units for Cid1, a portion of the DNA may be untranscribed, serving a regulatory or a structural function. For example, the DNA may itself comprise a promoter that is active in mammalian cells, or may utilize a promoter already present in the genome that is the transformation target. Such promoters include the CMV promoter, as well as the SV40 late promoter and retroviral LTRs (long terminal repeat elements), although many other promoter elements well known to the art may be employed in the practice of the invention.

Other elements functional in the host cells, such as introns, enhancers, polyadenylation sequences and the like, may also be a part of the DNA. Such elements may or may not be necessary for the function of the DNA, but may provide improved expression of the DNA by affecting transcription, stability of the MnRNA, or the like. Such elements may be included in the DNA as desired to obtain the optimal performance of the transforming DNA in the cell.

“Control sequences” is defined to mean DNA sequences necessary for the expression of an operably linked coding sequence in a particular host organism. The control sequences that are suitable for prokaryotic cells, for example, include a promoter, and optionally an operator sequence, and a ribosome binding site. Eukaryotic cells are known to utilize promoters, polyadenylation signals, and enhancers.

“Operably linked” is defined to mean that the nucleic acids are placed in a functional relationship with another nucleic acid sequence. For example, DNA for a presequence or secretory leader is operably linked to DNA for a peptide or polypeptide if it is expressed as a preprotein that participates in the secretion of the peptide or polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accord with conventional practice.

The DNA to be introduced into the cells further will generally contain either a selectable marker gene or a reporter gene or both to facilitate identification and selection of transformed cells from the population of cells sought to be transformed. Alternatively, the selectable marker may be carried on a separate piece of DNA and used in a co-transformation procedure. Both selectable markers and reporter genes may be flanked with appropriate regulatory sequences to enable expression in the host cells. Useful selectable markers are well known in the art and include, for example, antibiotic and herbicide-resistance genes, such as neo, hpt, dhfr, bar, aroA, dapA and the like. See also, the genes listed on Table 1 of Lundquist et al. (U.S. Pat. No. 5,848,956).

Reporter genes are used for identifying potentially transformed cells and for evaluating the functionality of regulatory sequences. Reporter genes which encode for easily assayable proteins are well known in the art. In general, a reporter gene is a gene which is not present in or expressed by the recipient organism or tissue and which encodes a protein whose expression is manifested by some easily detectable property, e.g., enzymatic activity. Preferred genes include the chloramphenicol acetyl transferase gene (cat) from Tn9 of E. coli, the beta-glucuronidase gene (gus) of the uidA locus of E. Coli, and the luciferase gene from firefly Photinus pyralis. Expression of the reporter gene is assayed at a suitable time after the DNA has been introduced into the recipient cells.

The general methods for constructing recombinant DNA which can transform target cells are well known to those skilled in the art, and the same compositions and methods of construction may be utilized to produce the DNA useful herein. For example, J. Sambrook et al., Molecular Cloning: A Laboratory Manual, Cold Spring Harbor Laboratory, NY (1989), provides suitable methods of construction.

2. Transformation into Host Cells

The recombinant DNA can be readily introduced into the host cells, e.g., mammalian, bacterial, yeast or insect cells by transfection with an expression vector comprising DNA encoding a human Cid1 polypeptide, or its complement, by any procedure useful for the introduction into a particular cell, e.g., physical or biological methods, to yield a transformed cell having the recombinant DNA stably integrated into its genome, so that the DNA molecules, sequences, or segments, of the present invention are expressed by the host cell.

Physical methods to introduce a DNA into a host cell include calcium phosphate precipitation, lipofection, particle bombardment, microinjection, electroporation, and the like. Biological methods to introduce the DNA of interest into a host cell include the use of DNA and RNA viral vectors. The main advantage of physical methods is that they are not associated with pathological or oncogenic processes of viruses. However, they are less precise, often resulting in multiple copy insertions, random integration, disruption of foreign and endogenous gene sequences, and unpredictable expression. Viral vectors, and especially retroviral vectors, have become the most widely used method for inserting genes into mammalian, e.g., human cells. Other viral vectors can be derived from poxviruses, herpes simplex virus I, adenoviruses and adeno-associated viruses, and the like.

As used herein, the term “cell line” or “host cell” is intended to include well-characterized homogenous, biologically pure populations of cells. These cells may be eukaryotic cells that are neoplastic or which have been “immortalized” in vitro by methods known in the art, as well as primary cells, or prokaryotic cells. The cell line or host cell is preferably of mammalian origin, but cell lines or host cells of non-mammalian origin may be employed, including plant, insect, yeast, fungal or bacterial sources.

“Transfected” or “transformed” is used herein to include any host cell or cell line, the genome of which has been altered or augmented by the presence of at least one DNA sequence, which DNA is also referred to in the art of genetic engineering as “heterologous DNA,” “recombinant DNA,” “exogenous DNA,” “genetically engineered,” “non-native,” or “foreign DNA,” wherein said DNA was isolated and introduced into the genome of the host cell or cell line by the process of genetic engineering. The host cells of the present invention are typically produced by transfection with a DNA sequence in a plasmid expression vector, a viral expression vector, or as an isolated linear DNA sequence. Preferably, the transfected DNA is a chromosomally integrated recombinant DNA sequence, which comprises a gene encoding a Cid1 polypeptide of the invention or its complement, which host cell may or may not express significant levels of autologous or “native” Cid1 polypeptide.

To confirm the presence of the preselected DNA sequence in the host cell, a variety of assays may be performed. Such assays include, for example, “molecular biological” assays well known to those of skill in the art, such as Southern and Northern blotting, RT-PCR and PCR; and “biochemical” assays, such as detecting the presence or absence of a particular polypeptide, e.g., by immunological means (ELISAs and Western blots).

To detect and quantitate RNA produced from introduced preselected DNA segments, RT-PCR may be employed. In this application of PCR, it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique demonstrates the presence of an RNA species and gives information about the integrity of that RNA. The presence or absence of an RNA species can also be determined using dot or slot blot Northern hybridizations. These techniques are modifications of Northern blotting and only demonstrate the presence or absence of an RNA species.

While Southern blotting and PCR may be used to detect the preselected DNA segment in question, they do not provide information as to whether the preselected DNA segment is being expressed. Expression may be evaluated by specifically identifying the peptide products of the introduced preselected DNA sequences or evaluating the phenotypic changes brought about by the expression of the introduced preselected DNA segment in the host cell.

B. Polypeptides of the Invention

The present isolated, purified polypeptides, can be synthesized in vitro, e.g., by the solid phase peptide synthetic method or by recombinant DNA approaches (see above, and including in vitro transcription/translation systems). The polypeptides may be fusion polypeptides, i.e., the polypeptide comprises a portion of Cid1 and another peptide or polypeptide, e.g., a His tag. The solid phase peptide synthetic method is an established and widely used method, which is described in the following references: Stewart et al., Solid Phase Peptide Synthesis, W. H. Freeman Co., San Francisco (1969); Merrifield, J. Am. Chem. Soc., 85 2149 (1963); Meienhofer in “Hormonal Proteins and Peptides,” ed.; C. H. Li, Vol. 2 (Academic Press, 1973), pp. 48-267; Bavaay and Merrifield, “The Peptides,” eds. E. Gross and F. Meienhofer, Vol. 2 (Academic Press, 1980) pp. 3-285; and Clark-Lewis et al., Meth. Enzymol., 287, 233 (1997). These polypeptides can be further purified by fractionation on immunoaffinity or ion-exchange columns; ethanol precipitation; reverse phase HPLC; chromatography on silica or on an anion-exchange resin such as DEAE; chromatofocusing; SDS-PAGE; ammonium sulfate precipitation; gel filtration using, for example, Sephadex G-75; or ligand affinity chromatography.

Once isolated and characterized, derivatives, e.g., chemically derived derivatives, of a given polypeptide of the invention can be readily prepared. For example, amides of the polypeptide of the invention may also be prepared by techniques well known in the art for converting a carboxylic acid group or precursor, to an amide. A preferred method for amide formation at the C-terminal carboxyl group is to cleave the polypeptide from a solid support with an appropriate amine, or to cleave in the presence of an alcohol, yielding an ester, followed by aminolysis with the desired amine.

Salts of carboxyl groups of a polypeptide of the invention may be prepared in the usual manner by contacting the polypeptide with one or more equivalents of a desired base such as, for example, a metallic hydroxide base, e.g., sodium hydroxide; a metal carbonate or bicarbonate base such as, for example, sodium carbonate or sodium bicarbonate; or an amine base such as, for example, triethylamine, triethanolamine, and the like.

N-acyl derivatives of an amino group of the polypeptide may be prepared by utilizing an N-acyl protected amino acid for the final condensation, or by acylating a protected or unprotected peptide. 0-acyl derivatives may be prepared, for example, by acylation of a free hydroxy peptide or peptide resin. Either acylation may be carried out using standard acylating reagents such as acyl halides, anhydrides, acyl imidazoles, and the like. Both N- and O-acylation may be carried out together, if desired.

Formyl-methionine, pyroglutamine and trimethyl-alanine may be substituted at the N-terminal residue of the polypeptide. Other amino-terminal modifications include aminooxypentane modifications (see Simmons et al., Science, 276, 276 (1997)).

The polypeptides of the invention include polypeptides having amino acid substitutions, i.e., variant polypeptides. The variant polypeprides include the substitution of at least one amino acid residue in the polypeptide for another amino acid residue, including substitutions which utilize the D rather than L form, as well as other well known amino acid analogs, e.g., unnatural amino acids such as, -disubstituted amino acids, N-alkyl amino acids, lactic acid, and the like. These analogs include phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, omithine, citruline, -methyl-alanine, para-benzoyl-phenylalanine, phenylglycine, propargylglycine, sarcosine, —N,N,N-trimethyllysine, —N-acetyllysine, N-acetylserine, N-formylmethionine, 3-methylhistidine, 5-hydroxylysine, —N-methylarginine, and other similar amino acids and imino acids and tert-butylglycine.

Conservative amino acid substitutions are preferred—that is, for example, aspartic-glutamic as acidic amino acids; lysine/arginine/histidine as basic amino acids; leucine/isoleucine, methionine/valine, alanine/valine as hydrophobic amino acids; serine/glycine/alanine/threonine as hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide.

Acid addition salts of the polypeptide or of amino residues of the polypeptide may be prepared by contacting the polypeptide or amine with one or more equivalents of the desired inorganic or organic acid, such as, for example, hydrochloric acid. Esters of carboxyl groups of the peptides may also be prepared by any of the usual methods known in the art.

It is also envisioned that the polypeptide of the invention may comprise moieties, e.g., other peptide or polypeptide molecules (fusion polypeptides), such as antibodies or fragments thereof, nucleic acid molecules, sugars, lipids, e.g., cholesterol or other lipid derivatives which may increase membrane solubility, fats, a detectable signal molecule such as a radioisotope, e.g., gamma emitters, small chemicals, metals, salts, synthetic polymers, e.g., polylactide and polyglycolide, and surfactants which preferably are covalently attached or linked to polypeptide of the invention.

C. Antibodies of the Invention

The antibodies of the invention are prepared by using standard techniques. To prepare polyclonal antibodies or “antisera,” an animal is inoculated with an antigen that is an isolated and purified polypeptide of the invention, and immunoglobulins are recovered from a fluid, such as blood serum, that contains the immunoglobulins, after the animal has had an immune response. For inoculation, the antigen is preferably bound to a carrier peptide and emulsified using a biologically suitable emulsifying agent, such as Freund's incomplete adjuvant. A variety of mammalian or avian host organisms may be used to prepare polyclonal antibodies

Following immunization, Ig is purified from the immunized bird or mammal, e.g., goat, rabbit, mouse, rat, or donkey and the like. For certain applications, it is preferable to obtain a composition in which the antibodies are essentially free of antibodies that do not react with the immunogen. This composition is composed virtually entirely of the high titer, monospecific, purified polyclonal antibodies to the antigen. Antibodies can be purified by affinity chromatography. Purification of antibodies by affinity chromatography is generally known to those skilled in the art (see, for example, U.S. Pat. No. 4,533,630). Briefly, the purified antibody is contacted with the purified polypeptide, or a peptide thereof, bound to a solid support for a sufficient time and under appropriate conditions for the antibody to bind to the polypeptide or peptide. Such time and conditions are readily determinable by those skilled in the art. The unbound, unreacted antibody is then removed, such as by washing. The bound antibody is then recovered from the column by eluting the antibodies, so as to yield purified, monospecific polyclonal antibodies.

Monoclonal antibodies can be also prepared, using known hybridoma cell culture techniques. In general, this method involves preparing an antibody-producing fused cell line, e.g., of primary spleen cells fused with a compatible continuous line of myeloma cells, and growing the fused cells either in mass culture or in an animal species, such as a murine species, from which the myeloma cell line used was derived or is compatible. Such antibodies offer many advantages in comparison to those produced by inoculation of animals, as they are highly specific and sensitive and relatively “pure” immunochemically. Immunologically active fragments of the present antibodies are also within the scope of the present invention, e.g., the F(ab) fragment, scFv antibodies, as are partially humanized monoclonal antibodies.

Thus, it will be understood by those skilled in the art that the hybridomas herein referred to may be subject to genetic mutation or other changes while still retaining the ability to produce monoclonal antibody of the same desired specificity. The present invention encompasses mutants, other derivatives and descendants of the hybridomas.

It will be further understood by those skilled in the art that a monoclonal antibody may be subjected to the techniques of recombinant DNA technology to produce other derivative antibodies, humanized or chimeric molecules or antibody fragments which retain the specificity of the original monoclonal antibody. Such techniques may involve combining DNA encoding the immunoglobulin variable region, or the complementarity determining regions (CDRs), of the monoclonal antibody with DNA coding the constant regions, or constant regions plus framework regions, of a different immunoglobulin, for example, to convert a mouse-derived monoclonal antibody into one having largely human immunoglobulin characteristics (see EP 184187A, 2188638A, herein incorporated by reference).

The antibodies of the invention are useful for detecting or determining the presence or amount of a polypeptide of the invention in a sample. The antibodies are contacted with the sample for a period of time and under conditions sufficient for antibodies to bind to the polypeptide so as to form a binary complex between at least a portion of said antibodies and said polypeptide. Such times, conditions and reaction media can be readily determined by persons skilled in the art.

For example, the cells are lysed to yield an extract which comprises cellular proteins. Alternatively, intact cells are permeabilized in a manner which permits macromolecules, i.e., antibodies, to enter the cell. The antibodies of the invention are then incubated with the protein extract, e.g., in a Western blot, or permeabilized cells, e.g., prior to flow cytometry, so as to form a complex. The presence or amount of the complex is then determined or detected.

The antibodies of the invention may also be coupled to an insoluble or soluble substrate. Soluble substrates include proteins such as bovine serum albumin. Preferably, the antibodies are bound to an insoluble substrate, i.e., a solid support. The antibodies are bound to the support in an amount and manner that allows the antibodies to bind the polypeptide (ligand). The amount of the antibodies used relative to a given substrate depends upon the particular antibody being used, the particular substrate, and the binding efficiency of the antibody to the ligand. The antibodies may be bound to the substrate in any suitable manner. Covalent, noncovalent, or ionic binding may be used. Covalent bonding can be accomplished by attaching the antibodies to reactive groups on the substrate directly or through a linking moiety.

The solid support may be any insoluble material to which the antibodies can be bound and which may be conveniently used in an assay of the invention. Such solid supports include permeable and semipermeable membranes, glass beads, plastic beads, latex beads, plastic microtiter wells or tubes, agarose or dextran particles, sepharose, and diatomaceous earth. Alternatively, the antibodies may be bound to any porous or liquid permeable material, such as a fibrous (paper, felt etc.) strip or sheet, or a screen or net. A binder may be used as long as it does not interfere with the ability of the antibodies to bind the ligands.

The invention will be further described by reference to the following non-limiting example.

### EXAMPLE I

### Identification of Human Cid1 Homologs

A tblastn using the following parameters:

Expectation value (E): 10.0

Alignment view options: pariwise

Filter query sequences: yes

Cost to open a gap:

Cost to expand a gap:

X dropoff value for grabbed alignment:

Show GI's in deflines: No

Penalty for a nucleotide mismatch: −3

Reward for a nucleotide match: 1

Threshold for extending hits:

Perform gapped alignment: yes

Query Genetic code to use: standard

DB Genetic code: standard

Number of processors to use: 2

Believe the query define: no

Matrix: BLOSUM62

Word size:

Effective length of the database:

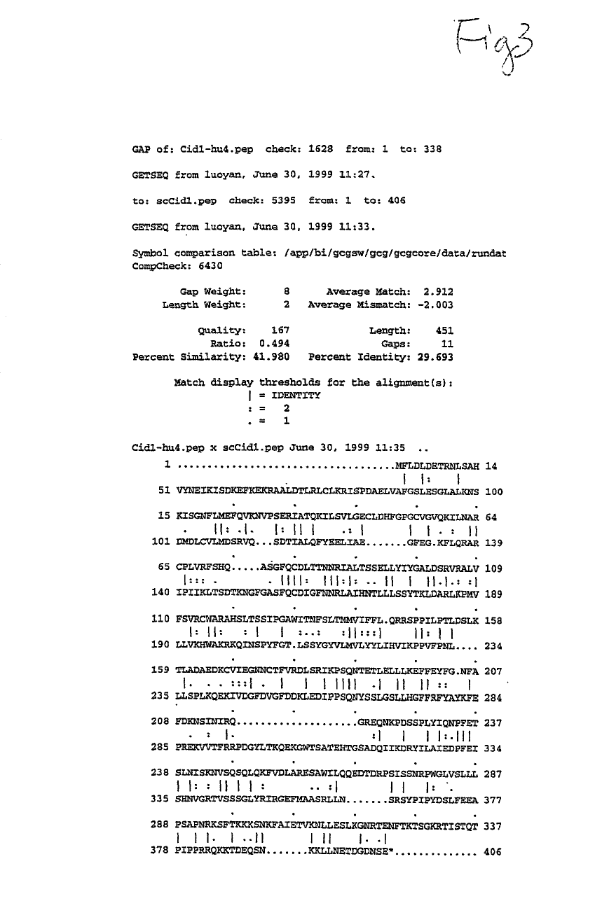

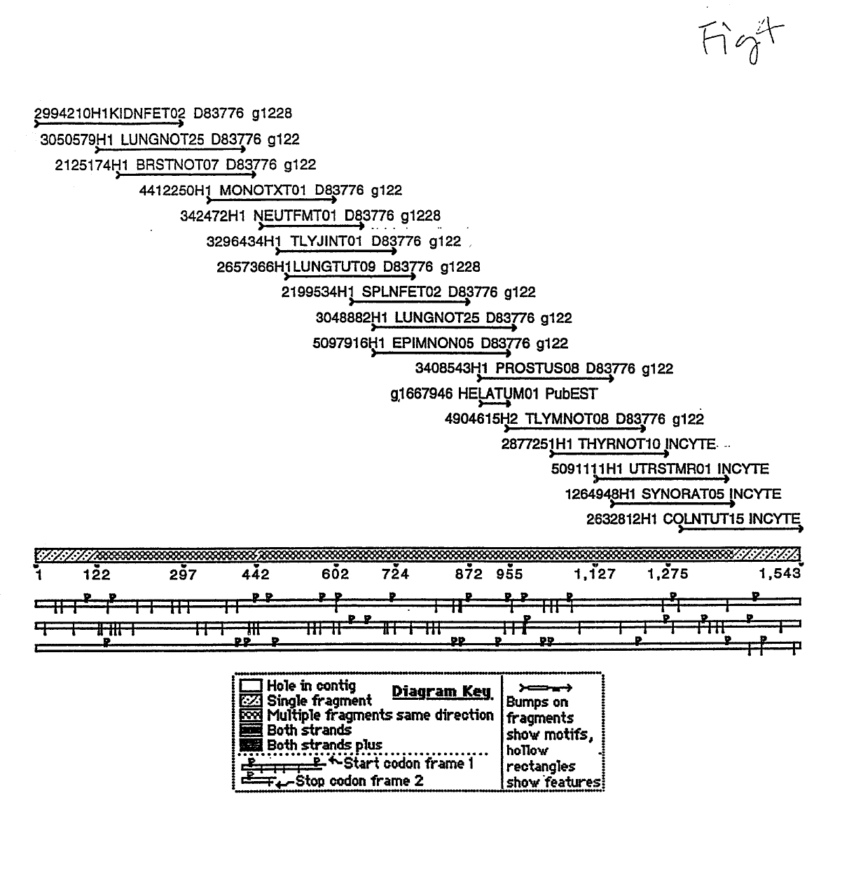

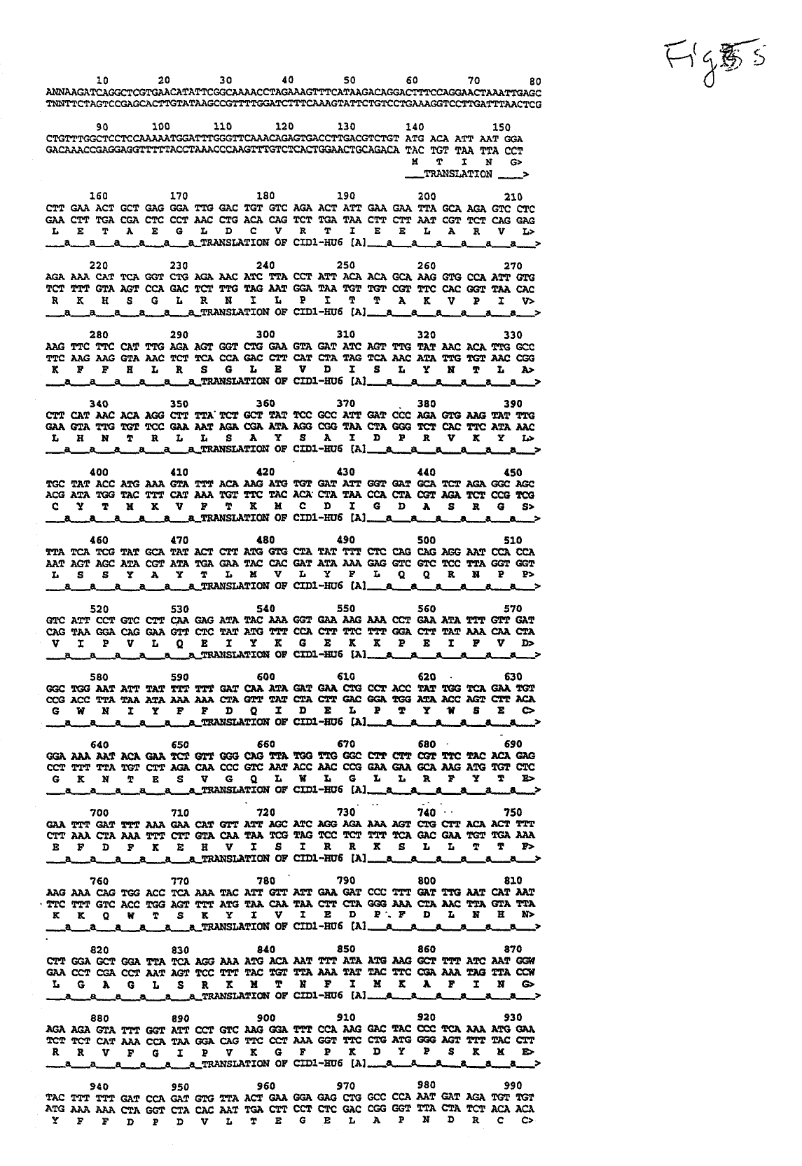

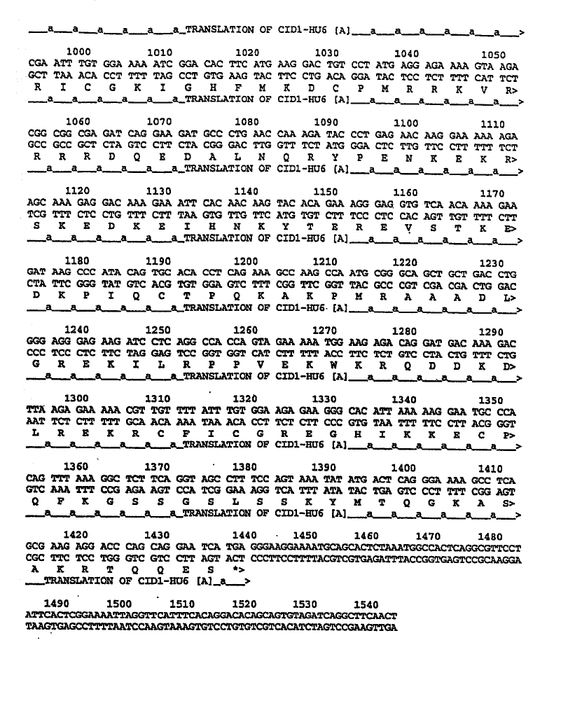

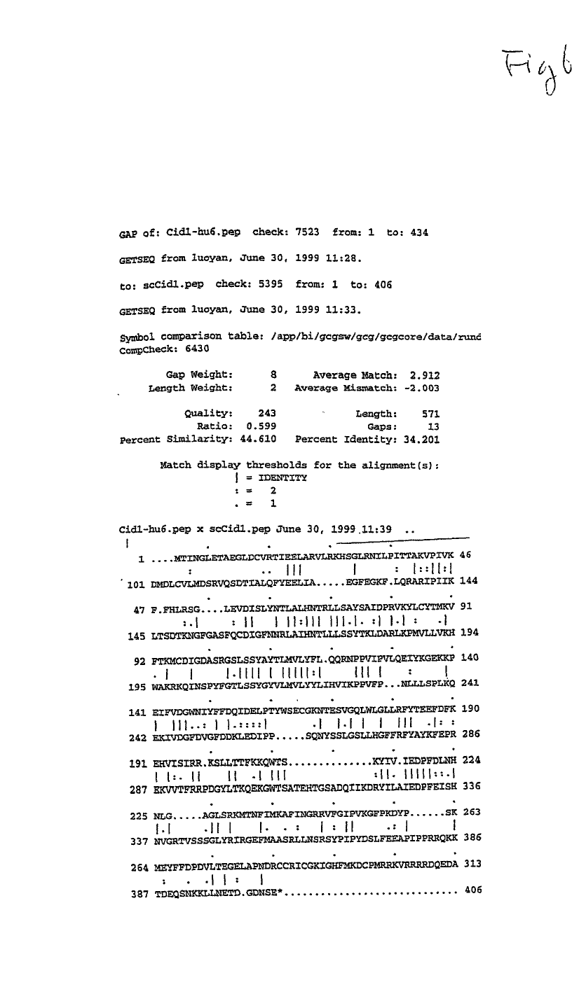

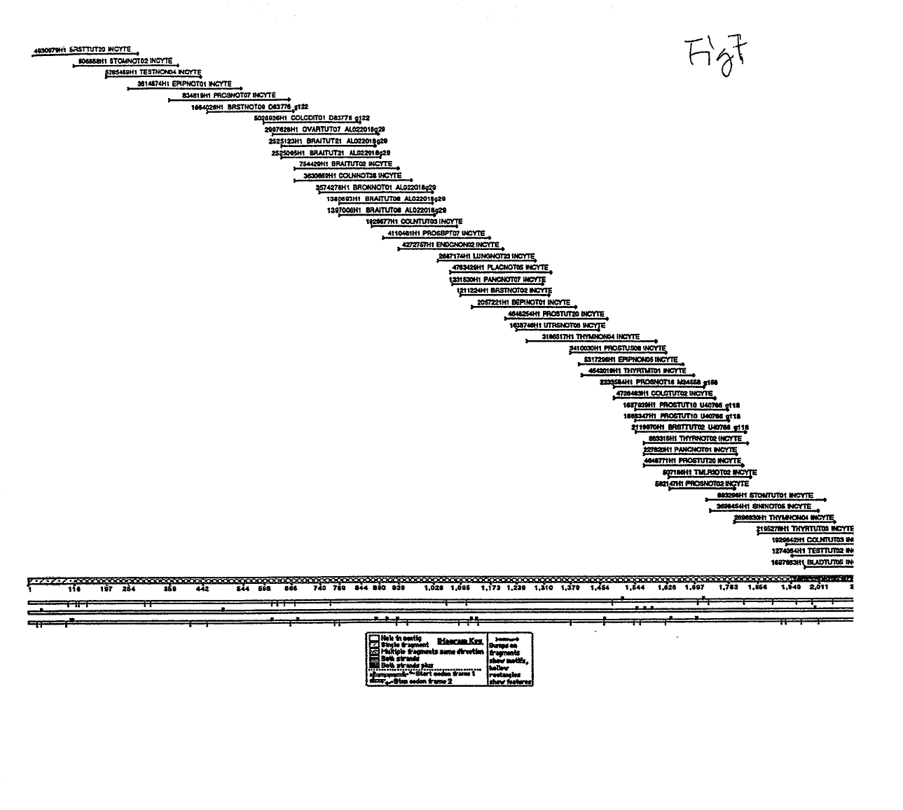

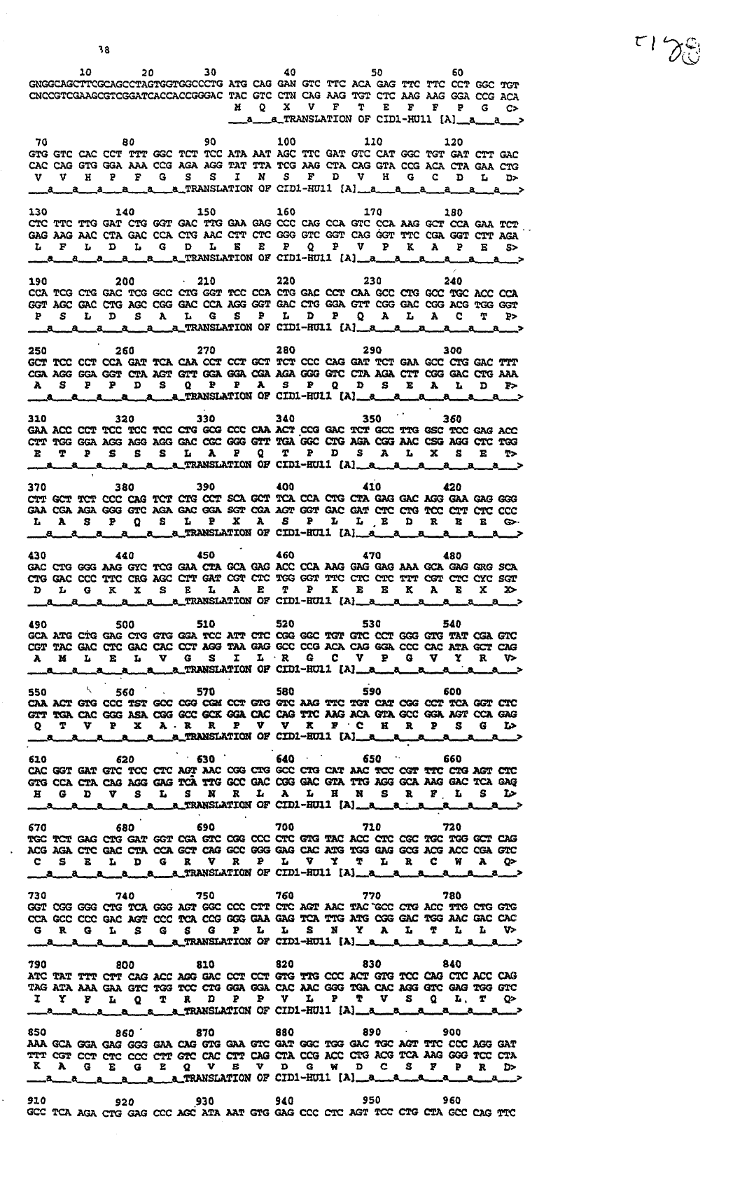

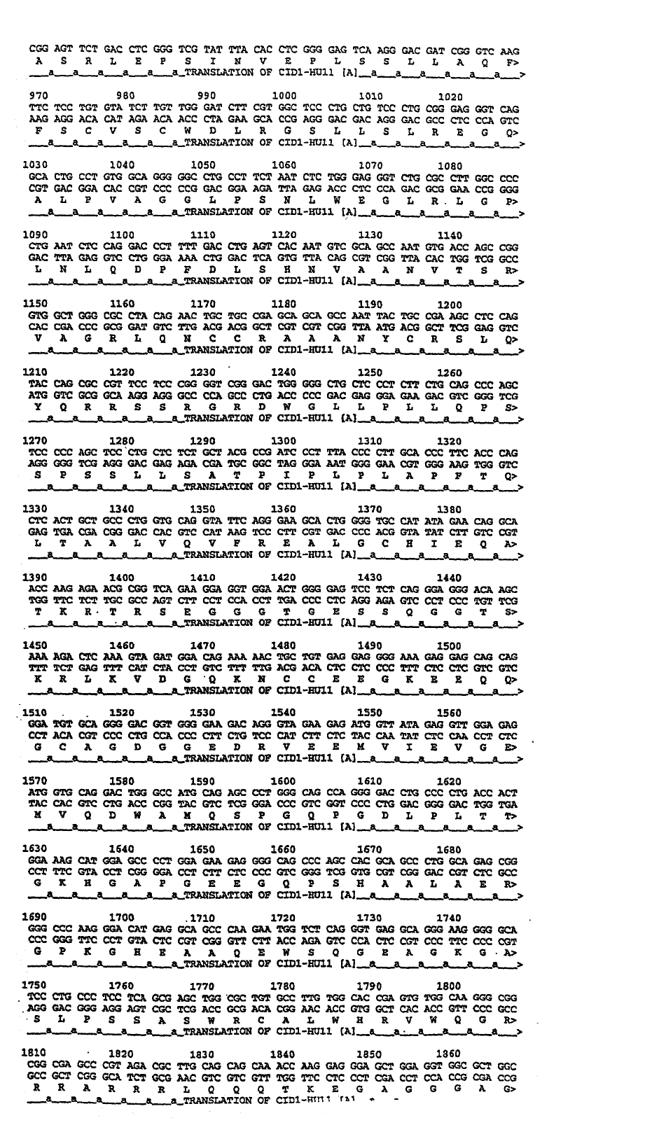

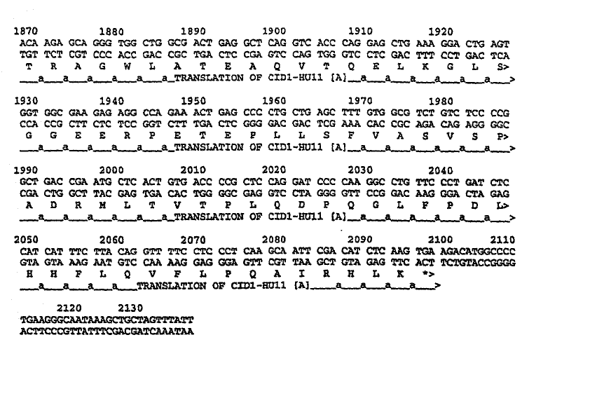

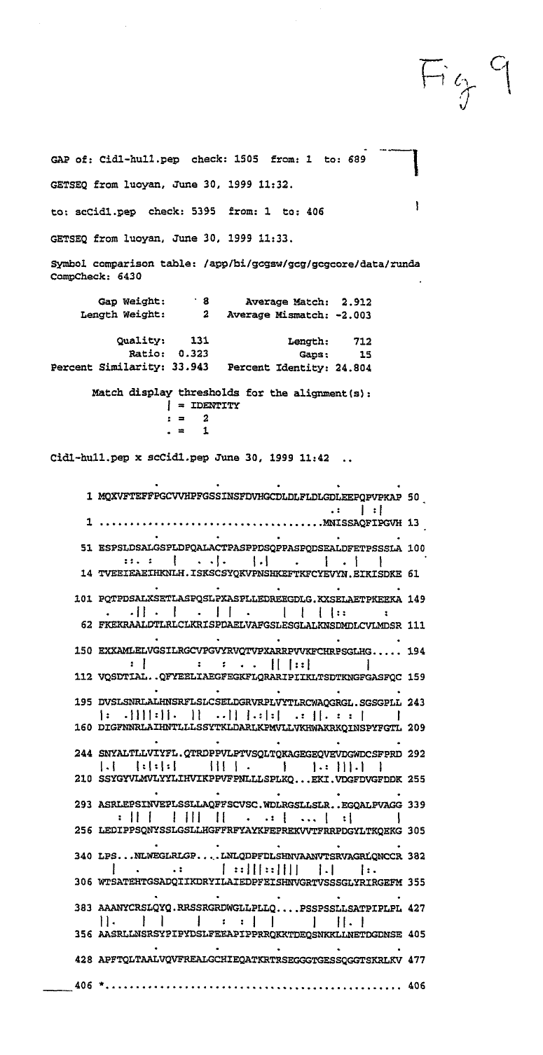

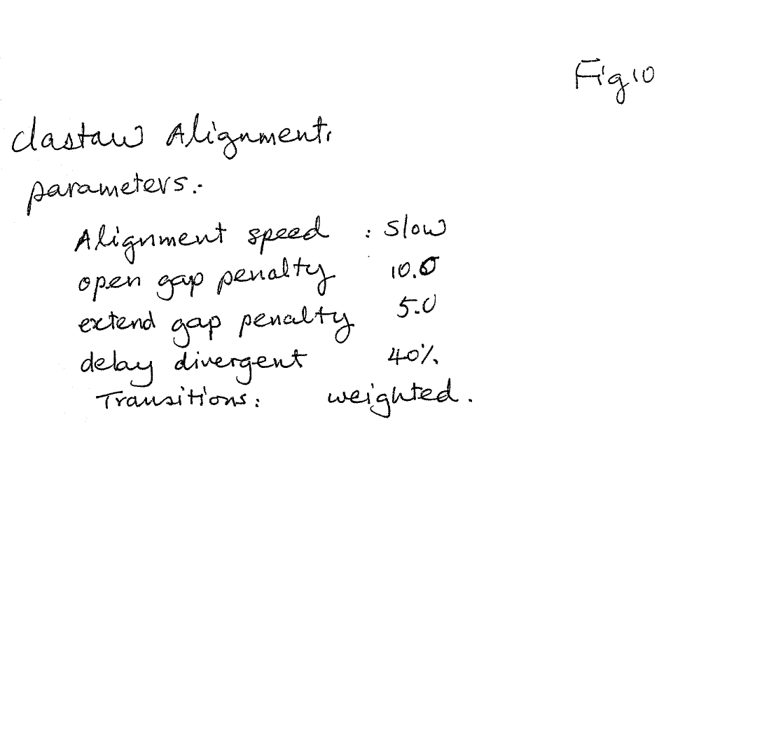

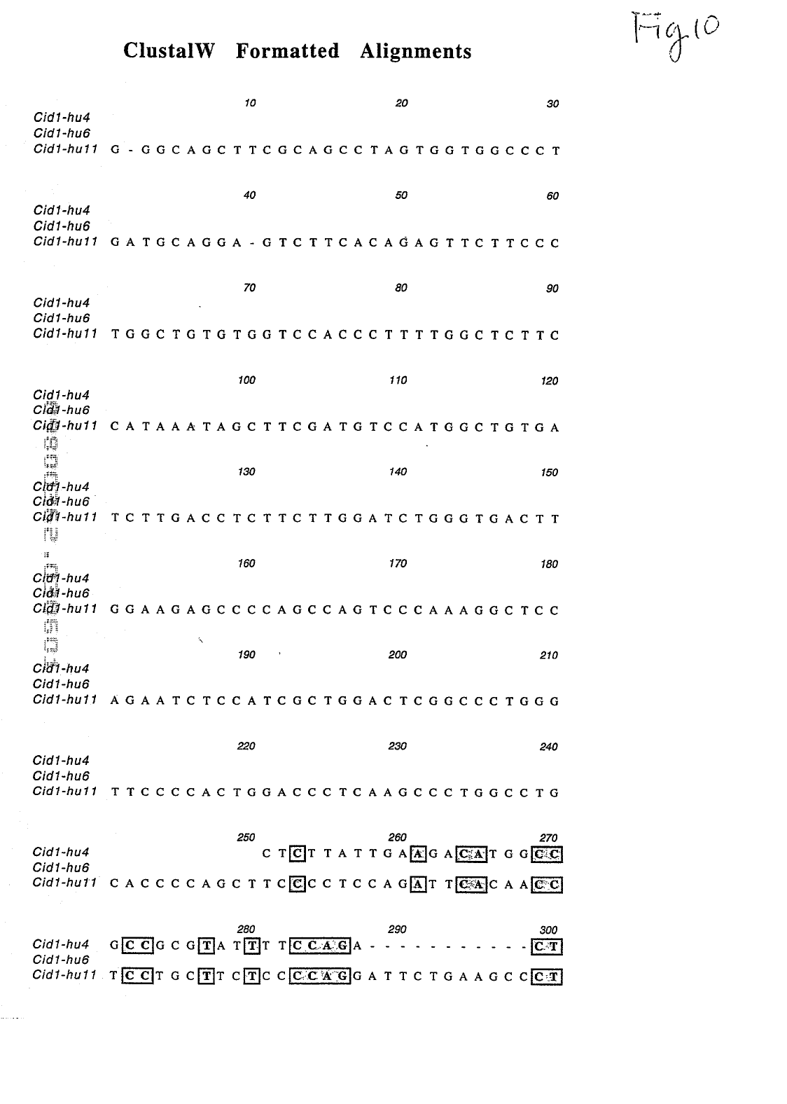

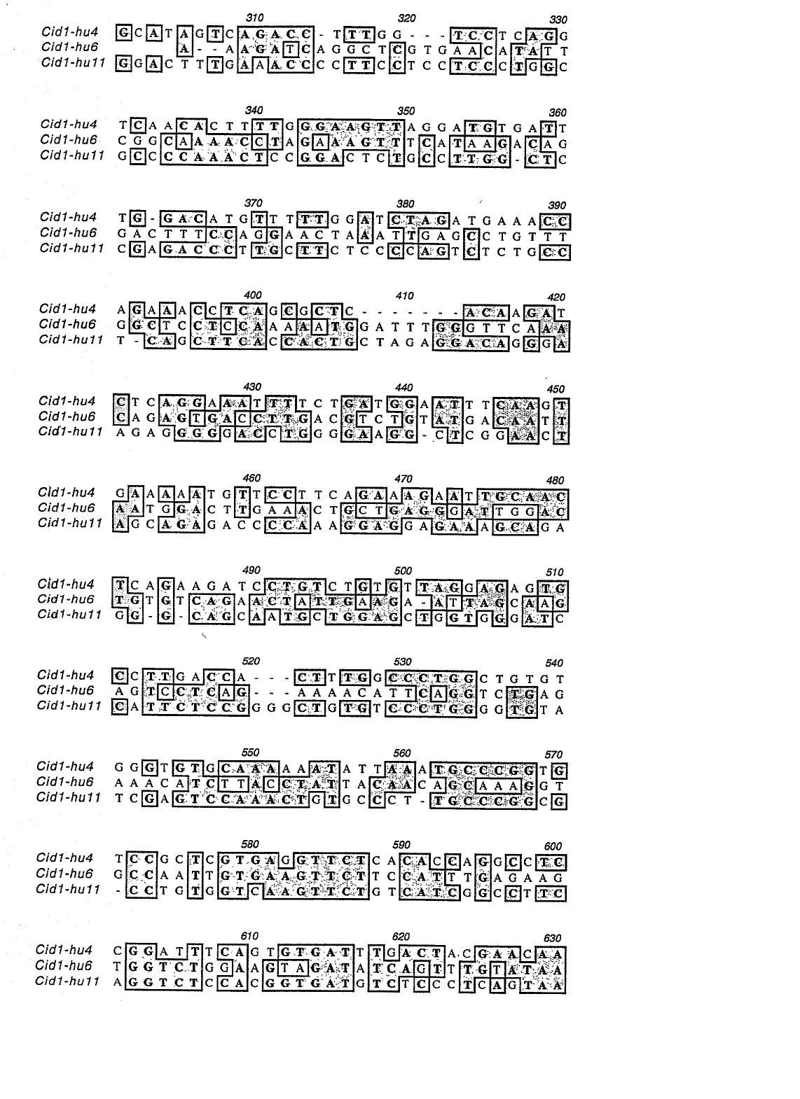

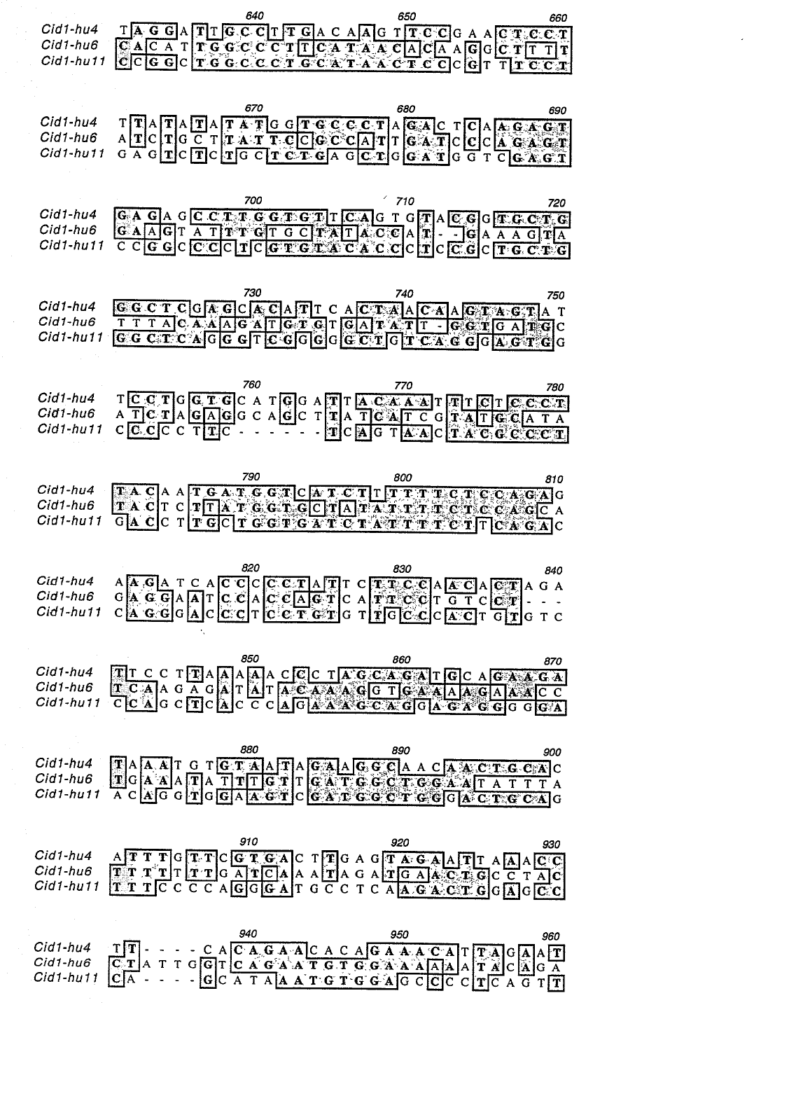

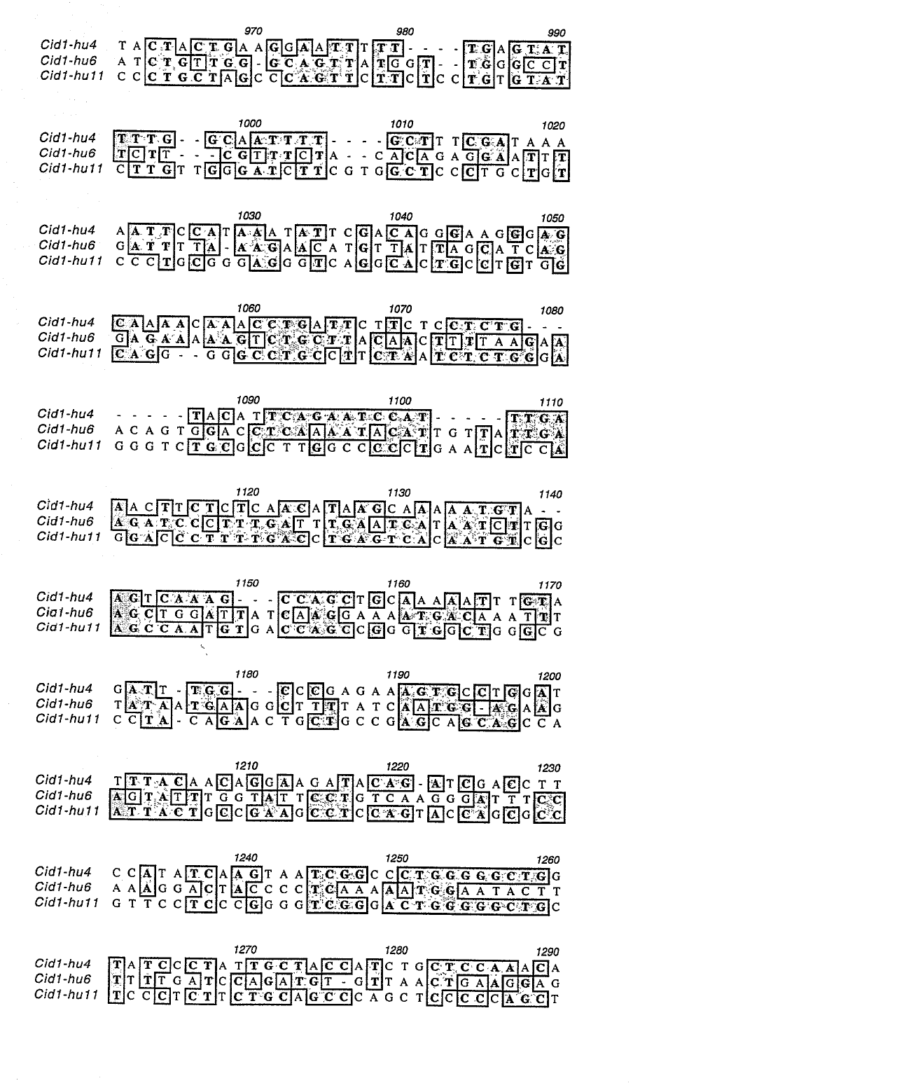

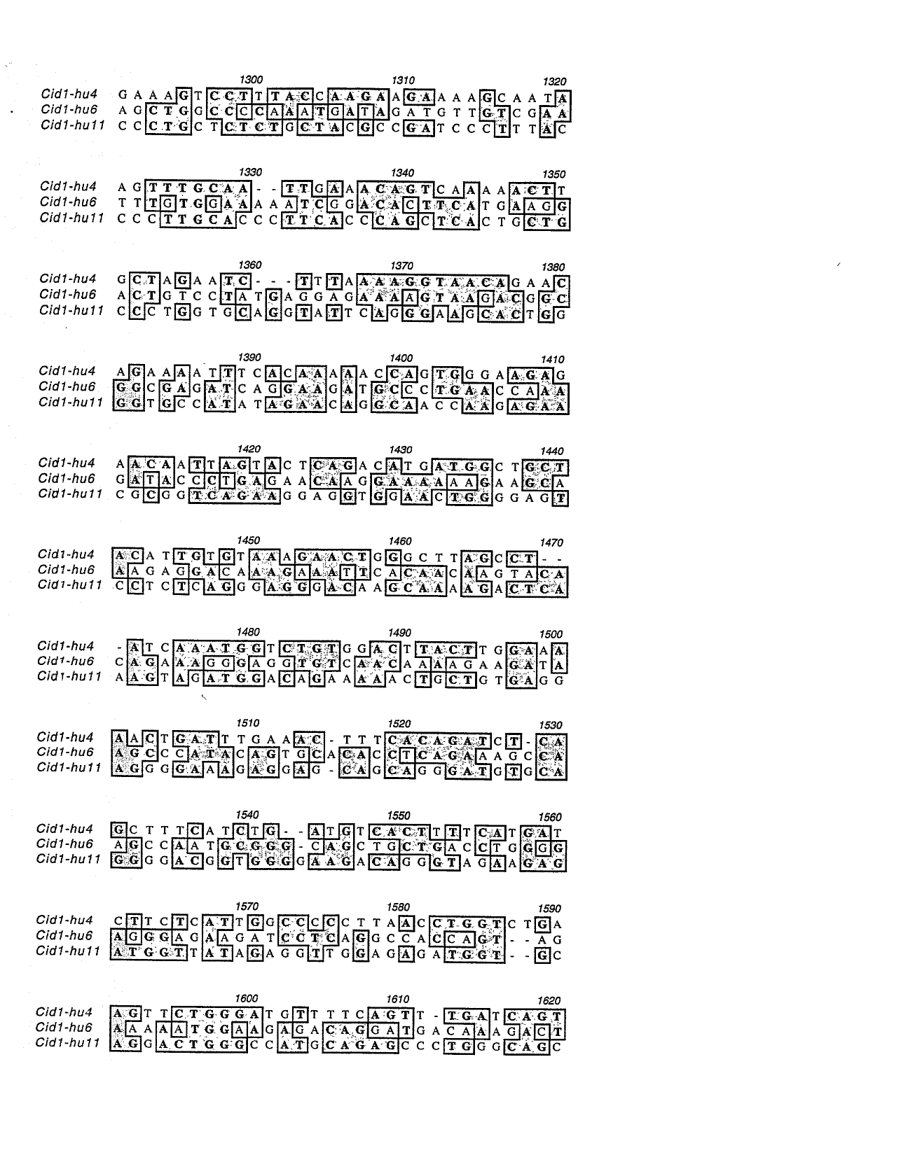

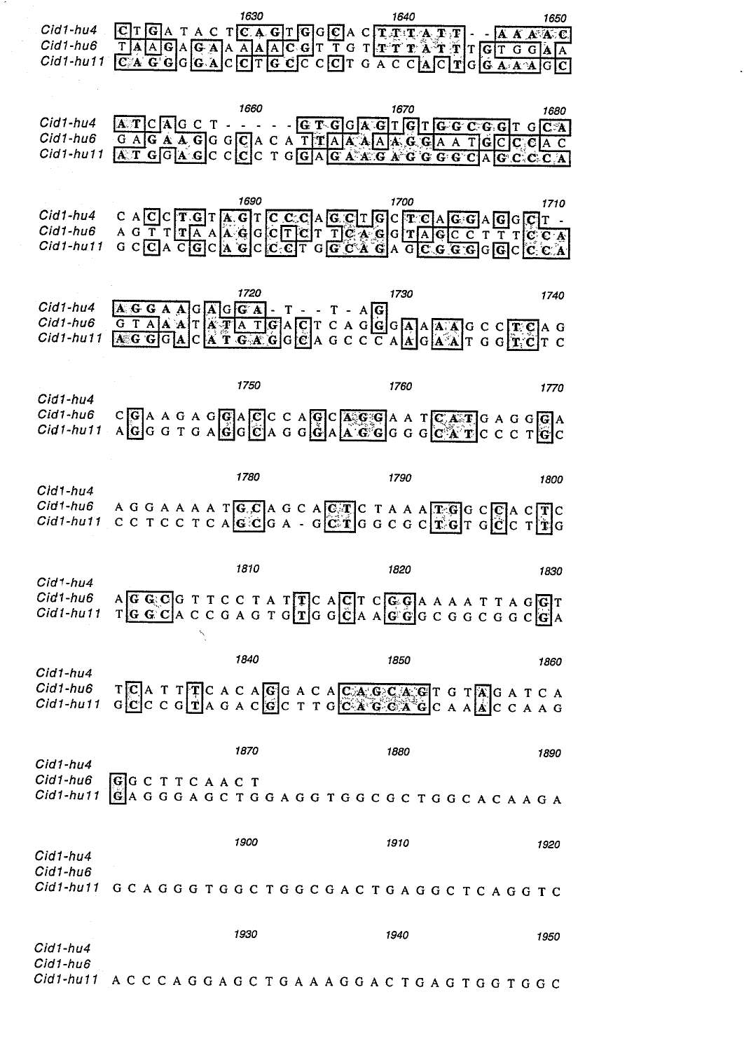

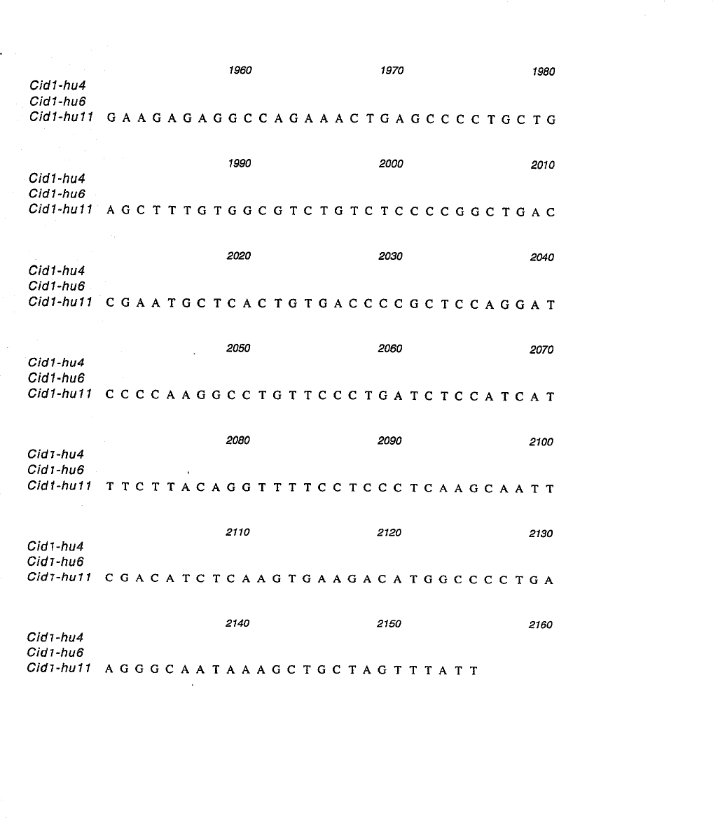

search was conducted of the Incyte database (Incyte46May) using Genbank clone Accession# AF105076 (Schizosaccharomyces pombe Cid1 gene) as the query. The resulting EST sequences were used as queries to do a second round of blast search. Three contiguous sequences were obtained: Cid1-hu4, Cid1-hu6 and Cid1-hu11 (SEQ ID NO: 1, SEQ ID NO:3, and SEQ ID NO:5, respectively). The putative open reading frames (ORFs) of the cDNAs for Cid1-hu4, Cid1-hu6 and Cid1-hu11 were translated into polypeptides (SEQ ID NO:2, SEQ ID NO:4, and SEQ ID NO:6, respectively; see FIGS. 2, 5 and 8) and aligned with Schizosaccharomyces pomhe Cid1 polypeptide sequences (see FIGS. 3, 6 and 9). Cid1-hu4 has 42% similarity/30% identity with the Schizosaccharomyces pombe Cid1 gene, and is expressed in 16 tumor libraries out of the 42 total libraries (38%) identified in the Incyte database (Table 1) (last two column labelled “Abund library” and “Percent Abund in Found” respectively). The tissue distribution for Cid1-hu4 is shown in Table 2.

TABLE 1

| | | | |
|---|---|---|---|
| GBLANOP01 | gallbladder, 25F, TIGR | 1 | 0.0591 |
| THP1NOB01 | periph blood, promonocyte line, | 1 | 0.0329 |
| | THP-1, AML, control | | |
| OVARTUT02 | ovary tumor, mucinous cystadenoma, | 1 | 0.0283 |
| | 51F | | |
| LIVRNOM01 | liver, 49M, WM | 1 | 0.0254 |
| OVARNOT03 | ovary, aw/mucinous cystadenoCA, | 1 | 0.0167 |
| | 43F, m/OVARTUT01 | | |
| UCMCL5T01 | umb cord blood, mononuclear cells, | 1 | 0.0084 |
| | t/IL-5 | | |
| LVENNOT01 | heart, left ventricle, 51F | 1 | 0.0456 |
| MONOTXS05 | periph blood, monocytes, 42F, | 1 | 0.0294 |
| | t/antilL-10, LPS, SUB | | |
| BRAITUT12 | brain tumor, frontal, astrocytoma, | 2 | 0.0275 |
| | 40F, m/BRAINOT14 | | |
| PROSTMT01 | prostate, AH, aw/adenoCA, | 1 | 0.0275 |
| | 67M | | |
| BRAHNOT01 | brain, hippocampus, 35M | 1 | 0.017 |
| LIVSFEM02 | liver/spleen, fetal, 20 w M, NORM, | 3 | 0.0079 |
| | WM | | |
| CONFNOT05 | fat, abdomen, 52F | 3 | 0.0863 |
| PROSTMT07 | prostate, AH, aw/adenoCA, | 1 | 0.0318 |
| | 73M | | |
| BRAINOT18 | brain, temporal, aw/mets malignant | 1 | 0.0303 |
| | melanoma, 34M | | |
| OVARDIN02 | ovary, endometriosis, aw/leiomyomata, | 1 | 0.0296 |
| | 39, NORM | | |
| COLNNOT19 | colon, cecum, aw/Crohn's, | 1 | 0.0293 |
| | 18F | | |
| THYMFET03 | thymus, fetal, M | 1 | 0.0279 |
| LUNGNOT15 | lung, aw/squamous cell CA, | 1 | 0.0277 |
| | 69M, m/LUNGTUT03 | | |
| SINTNOT21 | sm intestine, 8M | 1 | 0.0274 |
| LIVRTUT04 | liver tumor, hepatoma, 50M | 1 | 0.0272 |
| OVARNOT07 | ovary, aw/follicular cysts, 28F | 1 | 0.0269 |
| BRSTTUT13 | breast tumor, adenoCA, 46F, | 1 | 0.0265 |
| | m/BRSTNOT33 | | |
| BLADDIT01 | bladder, chronic cystitis, aw/urethral | 1 | 0.0263 |
| | adenoCA, 73M | | |
| PENCNOT09 | penis, corpora cavernosa, M | 1 | 0.0261 |
| BRAIFET01 | brain, fetal, 23 w M | 1 | 0.0258 |
| STOMFET01 | stomach, fetal, 20 w F | 1 | 0.0255 |
| KIDNTUT15 | kidney tumor, renal cell CA, | 1 | 0.0252 |
| | 65M m/KIDNNOT19 | | |
| OVARTUT04 | ovary tumor, TC CA, 53F | 1 | 0.0252 |
| LUNGNOT03 | lung, aw/mets thyroid CA, 79M, | 1 | 0.02 |
| | m/LUNGTUT02 | | |
| KIDNNOT32 | kidney, 49M | 1 | 0.0177 |
| SYNORAT03 | synovium, wrist, rheuA, 56F | 1 | 0.017 |
| BRAXNOT03 | brain, sensor-motor cortex, 35M | 1 | 0.0153 |
| SINTFET03 | sm intestine, fetal, 20 w F | 1 | 0.013 |
| THP1NOT03 | periph blood, promonocyte line, | 1 | 0.0129 |
| | THP-1, AML, untreated | | |
| DRGTNON04 | ganglion, dorsal root, thoracic, | 1 | 0.0113 |
| | aw/lymphoma, 32M, NORM | | |
| EOSIHET02 | periph blood, eosinophils, | 1 | 0.0106 |
| | hypereosinophilia, 48M | | |
| LIVSFEM02 | liver/spleen, fetal, 20 w M, NORM, | 4 | 0.0105 |
| | WM | | |
| LUNGFET03 | lung, fetal, 20 w F | 1 | 0.0091 |
| PANCNOT04 | pancreas, 5M | 1 | 0.0089 |
| BRAITUT02 | brain tumor, frontal, mets | 1 | 0.0075 |
| | hypemephroma, 58M | | |
| BRAINOM01 | brain, infant, 10 w F, NORM, WM | 1 | 0.0045 |

TABLE 2

| | | Tissue | % Abun | Usable in |
|---|---|---|---|---|
| Tissue Category | Found in | Abun | in Found | Found |
| Liver | 2/18 | 2 | 0.111 | 3931 |
| Genitalia, Female | 5/60 | 5 | 0.083 | 9514 |
| Nervous System | 8/115 | 8 | 0.070 | 13158 |
| Urinary Tract | 3/45 | 3 | 0.067 | 13404 |
| Hemic and Immune System | 7/114 | 7 | 0.061 | 14906 |
| Digestive System | 5/88 | 5 | 0.057 | 1692 |
| Pancreas | 1/19 | 1 | 0.053 | 11240 |
| Respiratory System | 3/61 | 3 | 0.049 | 19542 |
| Genitalia, Male | 3/79 | 3 | 0.038 | 3896 |
| Musculoskelatal System | 1/32 | 1 | 0.031 | 5895 |
| Connective Tissue | 1/33 | 1 | 0.030 | 3475 |
| Exocrine Glands | 1/43 | 1 | 0.023 | 3770 |
| Cardiovascular System | 1/59 | 1 | 0.017 | 2194 |

Cid1-hu6 has 44.6% similarity/34% identity with the Schizosaccharomyces pombe Cid1 gene, and is expressed in 46 tumor libraries out of the 126 total libraries (37%) identified in the Incyte database (Table 3). The tissue distribution for Cid1-hu6 is shown in Table 4.

TABLE 3

| | | | |
|---|---|---|---|
| MPHGNOT03 | periph blood, macrophages, adher | 20 | 0.2588 |
| | PBMC, M/F | | |
| BRAIDIT01 | brain, multiple sclerosis | 1 | 0.0272 |
| COLNTUT15 | colon tumor, adenoCA, 64F | 1 | 0.0247 |
| PITUNOT01 | pituitary, 16-70M/F, pool | 2 | 0.0223 |
| PROSTUT05 | prostate tumor, adenoCA, 69M, | 1 | 0.0145 |
| | m/PROSNOT07 | | |
| LUNGNOT23 | lung, aw/mets osteoSAR, 58M | 1 | 0.0126 |
| LUNGNOT25 | lung, aw/endobronchial carcinoid, | 1 | 0.0256 |
| | 33M | | |
| BRSTNOT07 | breast, PF changes, aw/adenoCA, | 1 | 0.0099 |
| | 43F | | |
| BONENOP01 | bone, aw/cancer, TIGR | 1 | 0.2571 |
| CARDFEP03 | heart, fetal, 8-10 w, pool, | 1 | 0.1309 |
| | BI | | |
| MUSLTDT02 | muscle, thigh, aw/lipoSAR, 64F | 1 | 0.1241 |
| AMLBNOT01 | periph blood, blast cells, AML, | 1 | 0.1063 |
| | 58F | | |
| PANCTUP01 | pancreatic tumor, TIGR | 1 | 0.0864 |
| MCLRUNT01 | PBMC, 60M, untreated | 5 | 0.0794 |
| HIPOAZT01 | brain, hippocampus, AD, 74M | 3 | 0.0742 |
| TLYMNOT04 | lymphocytes, activated Th1 cells, | 2 | 0.0664 |
| | 6-hr AB | | |
| KIDEUNC10 | kidney epithelial transf embryo line, | 1 | 0.0599 |
| | 293-EBNA, 1 g cDNA | | |
| MONOTXS05 | periph blood, monocytes, 42F, | 2 | 0.0588 |
| | t/antiL-10, LPS, SUB | | |
| EPIMNON05 | mammary, epithelial cells, 21F, | 2 | 0.0585 |
| | untreated NORM | | |
| CARDFEP01 | heart, fetal, 8-10 w, pool BI | 1 | 0.0531 |
| BRSTTUT22 | breast tumor, lobular CA, 59F, | 2 | 0.0529 |
| | m/BRSTNOR01, BRSTNOT16 | | |
| TESTNOT01 | testis, 37M | 1 | 0.048 |
| MUSCNOT01 | muscle, skeletal | 1 | 0.0445 |
| HELATXT05 | cervical tumor line, HeLa, adenoCA, | 1 | 0.044 |
| | 31F, t/Na butyrate 24 hr | | |
| LIVRTUS02 | liver tumor line, C3A, hepatoblastoma, | 1 | 0.0434 |
| | 15M, t/MCA, SUB | | |
| LUNGNOT39 | lung, asthma, 10M | 1 | 0.0398 |
| HELATUM01 | uterus, cervical tumor line, HeLa | 1 | 0.0353 |
| | S3, untreated, WM | | |
| SMCCNON03 | coronary artery, smooth muscle cells, | 1 | 0.0339 |
| | 3M, NORM | | |
| KIDETXS02 | kidney epithelial transf embryo line, | 1 | 0.0333 |
| | 293-EBNA, t/5AZA, SUB | | |
| THYMNOR02 | thymus, aw/congenital heart | 1 | 0.0333 |
| | abnormalities, 2F, RP | | |
| LNODNOT12 | lymph node, 11F | 1 | 0.0331 |
| TBLYNOT01 | T-B lymphoblast line, leukemia, | 1 | 0.0326 |
| | untreated | | |
| BRSTNOT31 | breast, aw/ductal adenoCA, | 1 | 0.0319 |
| | intraductal CA, 57F | | |
| LUNLTMT01 | lung, aw/adenoCA, 63F | 1 | 0.0315 |
| BRAWNOT01 | brain, dentate nucleus, 35M | 1 | 0.0311 |
| FETAFEM01 | fetus, 8-9 w, pool, NORM, | 2 | 0.0311 |
| | CGAP/WM | | |
| MUSCNOT07 | muscle, forearm, aw/intramuscular | 2 | 0.0308 |
| | hemangioma 38F | | |
| SINTNOT18 | sm intestine, aw/carcinoid, 59M | 1 | 0.0299 |
| LIVRTUT13 | liver tumor, mets neuroendocrine | 2 | 0.0296 |
| | CA, 62F | | |
| KIDNNOT26 | kidney, medulla/cortex, aw/renal | 1 | 0.0294 |
| | cell CA, 53F, m/KIDNTUT16 | | |
| PLACNOT05 | placenta, fetal, 18 w M | 1 | 0.0288 |
| SYNORAT05 | synovium, knee, rheuA, 62F | 1 | 0.0286 |
| PENGNOT01 | penis, glans, aw/scrotal urothelial | 1 | 0.0284 |
| | CA, 3M | | |
| SMCCNOS01 | coronary artery, smooth muscle | 1 | 0.0284 |
| | cells, 3M, t/TNF, IL-1, SUB | | |
| BRALNOT01 | brain, thalamus, 35M | 1 | 0.0278 |
| COLNNOT39 | colon, 16M | 1 | 0.0275 |
| SMCBUNT01 | bronchus, smooth muscle cells, | 1 | 0.0275 |
| | 21M, untreated | | |
| PROSTUT13 | prostate tumor, adenoCA, 59M, | 1 | 0.0271 |
| | m/PROSNOT19 | | |
| MONOTXT01 | periph blood, monocytes, 42F, | 1 | 0.0268 |
| | t/antiIL-10, LPS | | |
| BRSTNOT28 | breast, PF changes, 40F | 1 | 0.0267 |
| UTRSNOT08 | uterus, endometrium, aw/endometrial | 1 | 0.0267 |
| | polyp, 35F | | |
| THYRTMT01 | thyroid, aw/medullary CA, 56M | 1 | 0.0267 |
| GBLADIT01 | gallbladder, cholecystitis, | 1 | 0.0267 |
| | cholelithiasis, 18F | | |
| BRSTNOT32 | breast, NF breast disease, 46F | 1 | 0.0265 |
| BRSTTUT13 | breast tumor, adenoCA, 46F, | 1 | 0.0265 |
| | m/BRSTNOT33 | | |
| LIVRTUT09 | liver tumor line, C3A, hepatoblastoma, | 1 | 0.0263 |
| | 15M, untreated | | |
| OVARDIT01 | ovary, endometriosis, aw/leiomyomata, | 1 | 0.026 |
| | 39F | | |
| PROSNOT28 | prostate, AH, aw/adenoCA, 55M, | 1 | 0.026 |
| | m/PROSTUT16 | | |
| LUNGTUT07 | lung tumor, squamous cell CA, 50M | 1 | 0.0259 |
| STOMFET02 | stomach, fetal, 18 w M | 1 | 0.0259 |
| LIVRDIR01 | liver, primary biliary cirrhosis, | 2 | 0.0257 |
| | 63F, RP | | |
| PENCNOT10 | penis, corpora cavernosa, M | 1 | 0.0257 |
| LUNGNOT25 | lung, aw/endobronchial carcinoid, | 1 | 0.0256 |
| | 33M | | |
| ADRENOT11 | adrenal gland, aw/pheochromocytoma, | 1 | 0.0255 |
| | 43F, m/ADRETUT07 | | |
| TESTNOC01 | testis, 10-61M, pool, 1 g cDNA | 1 | 0.0255 |
| BRSTNOT09 | breast, aw/adenoCA, 45F, m/BRSTTUT08 | 1 | 0.0255 |
| ADRENOT15 | adrenal glands, fetal, 16 w F | 1 | 0.0254 |
| LUNLTUT02 | lung tumor, squamous cell CA, 64F | 1 | 0.0254 |
| THYMNOT04 | thymus, 3M | 1 | 0.0252 |
| PROSTUS08 | prostate tumor, adenoCA, 59M, SUB, | 1 | 0.025 |
| | m/PROSTNOT19 | | |
| SMCCNOT02 | coronary artery, smooth muscle | 1 | 0.025 |
| | cells, 3M, t/TNF, IL-1 | | |
| UTRSTUT04 | uterus tumor, leiomyoma, 34F | 1 | 0.025 |
| BRSTNOT19 | breast, aw/lobular CA, 67F | 1 | 0.0249 |
| MENITUT03 | brain tumor, benign meningioma, 35F | 1 | 0.0249 |
| LIVRTUT12 | liver tumor line, C3A, hepatoblastoma, | 1 | 0.0246 |
| | 15M, t/MCA 48 hr | | |
| OVARNOT12 | ovary, aw/leiomyomata, 36F | 1 | 0.0245 |
| SININOT04 | sm intestine, ileum, Crohn's, 26M | 1 | 0.0241 |
| OVARTUT03 | ovary tumor, seroanaplastic CA, 52F | 1 | 0.0235 |
| OVARNOT09 | ovary, follicular cysts, 28F | 1 | 0.0234 |
| TLYJINT01 | Jurkat line, T-cell leukemia, M, | 1 | 0.0227 |
| | t/PMA | | |
| UTRSTIMR01 | uterus, myometrium, aw/leiomyoma, | 2 | 0.0223 |
| | 41F, RP, m/UTRSTUT05 | | |
| LIVSFEM03 | liver/spleen, fetal, 20 w M, NORM, WM | 1 | 0.0214 |
| ENDCNOT03 | microvascular, dermal, endothelial | 1 | 0.021 |
| | cells, neonatal, M | | |
| BRSTNOM02 | breast, F, NORM, WM | 1 | 0.0207 |
| SCORNOT01 | spinal cord, 71M | 1 | 0.0201 |
| BRAINOT22 | brain, temporal, aw/neuroepithelial | 1 | 0.0199 |
| | tumor, 45M | | |
| LNODNOT11 | lymph node, 16 m M | 1 | 0.0191 |
| LUNGTUT02 | lung tumor, mets thyroid CA, 79M, | 1 | 0.0189 |
| | m/LUNGNOT03 | | |
| BRAIDIT05 | brain, acute/chronic multiple | 1 | 0.0183 |
| | sclerosis, pool | | |
| BRAINOM01 | brain, infant, 10 w F, NORM, WM | 4 | 0.0178 |
| NEUTFMT01 | periph blood, granulocytes, M/F, | 1 | 0.0175 |
| | t/fMLP | | |
| HNT2NOT01 | teratoCA line, hNT2, untreated | 1 | 0.0173 |
| TLYMNOT03 | lymphocytes, nonactivated Th1 cells | 1 | 0.0168 |
| LUNGNOT30 | lung, aw/Patau's syndrome, fetal, | 1 | 0.016 |
| | 20 w M | | |
| LUNGTUT03 | lung tumor, squamous cell CA, 69M, | 1 | 0.0159 |
| | m/LUNGNOT15 | | |
| LUNGFEM01 | lung, fetal, 19 w, NORM, WM | 1 | 0.0149 |
| BRSTNOT03 | breast, PF changes, aw/adenoCA, 54F, | 1 | 0.0147 |
| | m/BRSTTUT02 | | |
| TESTNOM01 | testis, M, NORM, CGAP/WM | 1 | 0.0147 |
| PROSTUT05 | prostate tumor, adenoCA, 69M, | 1 | 0.0145 |
| | m/PROSNOT07 | | |
| BEPINOT01 | bronchial epithelium primary line, | 1 | 0.0144 |
| | 54M, untreated | | |
| COLNFET02 | colon, fetal, 20 w F | 1 | 0.0143 |
| KIDNNOT19 | kidney, aw/renal cell CA, 65M, | 1 | 0.0143 |
| | m/KIDNTUT15 | | |
| THYRNOT03 | thryoid, aw/follicular adenoma, 28F | 1 | 0.0138 |
| SMCANOT01 | aortic smooth muscle line, M | 1 | 0.0136 |
| TESTTUT02 | testis tumor, embryonal CA, 31M | 1 | 0.0135 |
| TESTNOT03 | testis, 37M | 1 | 0.0129 |
| KIDNFET01 | kidney, fetal, 17 w F | 1 | 0.0128 |
| SPLNFET02 | spleen, fetal, 23 w M | 1 | 0.0126 |
| DRGLNOT01 | ganglion, dorsal root, thoracic/lumbar, | 1 | 0.0123 |
| | aw/lymphoma, 32M | | |
| THYRNOT10 | thyroid, lymphocytic thyroiditis, | 1 | 0.0123 |
| | aw/papillary CA, 30F | | |
| EOSITXT01 | periph blood, eosinophils, t/IL-5 | 1 | 0.0111 |
| DRGCNOT01 | ganglion, dorsal root, cervical, | 1 | 0.0104 |
| | aw/lymphoma, 32M | | |
| TONLNOP01 | tonsil, B-lymphocytes, germinal, | 1 | 0.0102 |
| | aw/tonsillitis, NORM, CGAP | | |
| TLYMNOT08 | T-lymphocytes, allogenic anergic, | 1 | 0.0101 |
| | 40-50M, t/OKT3 3 day | | |
| BRABDIR01 | brain, cerebellum, Huntington's | 1 | 0.0096 |
| | disease, aw/CVA, 57M, RP | | |
| PROSNON01 | prostate, 28M, NORM | 1 | 0.0094 |
| PANCTUT02 | pancreatic tumor, anaplastic CA, 45F | 1 | 0.0086 |
| UCMCL5T01 | umb cord blood, mononuclear cells, | 1 | 0.0084 |
| | t/IL-5 | | |
| CARDFEP02 | heart, fetal, 8-10 w, pool, BI | 1 | 0.0081 |
| UTRSNOT02 | uterus, aw/ovarian follicular cysts, | 1 | 0.0078 |
| | 34F | | |
| BRAITUT02 | brain tumor, frontal, mets | 1 | 0.0075 |
| | hypernephroma, 58M | | |
| NGANNOT01 | neuroganglion tumor, ganglioneuroma, | 1 | 0.0073 |
| | 9M | | |
| PLACNOM02 | placenta, neonatal, F, NORM, WM | 1 | 0.0056 |

TABLE 4

| | | Tissue | % Abun in | Usable in |
|---|---|---|---|---|
| Tissue Category | Found In | Abun | Found | Found |
| Endocrine System | 6/39 | 6 | 0.154 | 8979 |
| Respiratory System | 12/61 | 12 | 0.196 | 7959 |
| Genitalia, Male | 13/79 | 13 | 0.164 | 6898 |
| Digestive System | 7/88 | 7 | 0.080 | 4051 |
| Hemic and Immune System | 20/114 | 20 | 0.175 | 7727 |
| Nervous System | 15/115 | 15 | 0.130 | 3670 |
| Exocrine Glands | 11/43 | 11 | 0.256 | 10097 |
| Respiratory System | 1/61 | 1 | 0.016 | 3907 |
| Liver | 5/18 | 5 | 0.278 | 24724 |
| Embryonic Structures | 3/15 | 3 | 0.200 | 27903 |
| Genitalia, Female | 10/60 | 10 | 0.167 | 51142 |
| Musculoskeletal System | 5/32 | 5 | 0.156 | 13438 |
| Cardiovascular System | 8/59 | 8 | 0.136 | 37613 |
| Urinary Tract | 5/45 | 5 | 0.111 | 22902 |
| Pancreas | 2/19 | 2 | 0.105 | 12757 |

Cid1-hu11 has 34% similarity/25% identity with the whole molecule and 43% similarity/33% identity with the C-terminal half of Schizosaccharomyces pombe Cid1 gene, and is expressed in 29 tumor libraries out of the 50 total libraries (58%) identified in the Incyte database (Table 5). The tissue distribution for Cid1-hu11 is shown in Table 6.

TABLE 5

| | | | |
|---|---|---|---|
| STOMNOT02 | stomach, aw/adenoCA, 52M, | 1 | 0.0308 |
| | m/STOMTUT01 | | |
| BRSTTUT20 | breast tumor, ductal adenoCA, | 1 | 0.0257 |
| | 66F | | |
| BRAINOP02 | brain, infant, F, TIGR | 1 | 0.0558 |
| HNT2NOM01 | teratoCA line, hNT2, t/RA, WM | 1 | 0.0416 |
| PROSNOT07 | prostate, AH, aw/adenoCA, 69M, | 1 | 0.0348 |
| | m/PROSTUT05 | | |
| BRAITUT21 | brain tumor, frontal, meningioma, | 2 | 0.0316 |
| | 61F | | |
| BRAITUT08 | brain tumor, frontal, astrocytoma, | 2 | 0.0293 |
| | 47M | | |
| COLCDIT01 | colon, cecum, benign familial | 1 | 0.0285 |
| | polyposis, 16M | | |
| BRONNOT01 | bronchus, 15M | 1 | 0.0278 |
| BRSTNOM01 | breast, F, NORM, WM | 1 | 0.0264 |
| BRSTNOT09 | breast, aw/adenoCA, 45F, | 1 | 0.0255 |
| | m/BRSTTUT08 | | |
| EPIPNOT01 | prostate, epithelial cells, | 1 | 0.0252 |
| | 17M, untreated | | |
| OVARTUT07 | ovary tumor, adenoCA, 58F | 1 | 0.0228 |
| COLNNOT38 | colon, Patau's syndrome, fetal, | 1 | 0.0179 |
| | 20 w M | | |
| HEAANOT01 | heart, coronary artery, CAD, 46M | 1 | 0.0089 |
| BRAITUT02 | brain tumor, frontal, mets | 1 | 0.0075 |
| | hypernephroma, 58M | | |
| ENDCNON02 | heart coronary artery endothelial | 1 | 0.0743 |
| | cells, 3M, untreated, NORM | | |
| PROSTUT20 | prostate tumor, adenoCA, 58M | 2 | 0.0531 |
| THYMNON04 | thymus, 3M, NORM | 2 | 0.0513 |
| EPIPNON05 | prostate, epithelial cells, 17M, | 1 | 0.0486 |
| | untreated, NORM | | |
| PROSNOT02 | prostate, AH, aw/adenoCA, 50M, | 1 | 0.0435 |
| | m/PROSTUT01 | | |
| COLNTUT03 | colon tumor, sigmoid, adenoCA, | 2 | 0.0392 |
| | 62M, m/COLNNOT16 | | |
| STOMTUT01 | stomach tumor, adenoCA, 52M, | 1 | 0.0368 |
| | m/STOMNOT02 | | |
| ESOGTUN01 | esophageal tumor, adenoCA, 61M, | 1 | 0.0355 |
| | NORM | | |
| COLCTUT02 | colon tumor, cecum, carcinoid, | 1 | 0.0305 |
| | 30F | | |
| THYRNOT02 | thyroid, hyperthyroidism, 16F | 1 | 0.0303 |
| PROSTUT10 | prostate tumor, adenoCA, 66M, | 2 | 0.0289 |
| | m/PROSNOT15 | | |
| PLACNOT05 | placenta, fetal, 18 w M | 1 | 0.0288 |
| SINTNOT25 | sm intestine, 13M | 1 | 0.0283 |
| UTRSNOT06 | uterus, myometrium, atypical | 1 | 0.0282 |
| | hyperplasia, aw/adenoCA, 50F | | |
| PROSBPT07 | prostate, AH, aw/adenoCA, 53M | 1 | 0.0278 |
| THYRTUT03 | thyroid tumor, follicular | 1 | 0.0277 |
| | adenoma, 17M | | |
| OVARNOT07 | ovary, aw/follicular cysts, 28F | 1 | 0.0269 |
| BLADTUT05 | bladder tumor, TC CA, 66M, | 1 | 0.0268 |
| | m/BLADNOT06 | | |
| THYRTMT01 | thyroid, aw/medullary CA, 56M | 1 | 0.0267 |
| SININOT05 | sm intestine, ileum, aw/carcinoid, | 1 | 0.0262 |
| | 30F | | |
| PROSTMT03 | prostate, aw/adenoCA, 68M, | 1 | 0.0256 |
| | m/PROSTUT18 | | |
| LUNGTUT09 | lung tumor, squamous cell CA, 68M | 1 | 0.0253 |
| PROSTUS08 | prostate tumor, adenoCA, 59M, SUB, | 1 | 0.025 |
| | m/PROSNOT19 | | |
| TMLR3DT02 | periph blood, lymphocytes, non-adher | 1 | 0.0246 |
| | PBMC, M/F, 72-hr t/MLR | | |
| PANCNOT01 | pancrease, 29M | 1 | 0.0214 |
| BRAITUT21 | brain tumor, frontal, meningioma, | 1 | 0.0158 |
| | 61F | | |
| LUNGFEM01 | lung, fetal, 19 w, NORM, WM | 1 | 0.0149 |
| BEPINOT01 | bronchial epithelium primary line, | 1 | 0.0144 |
| | 54M, untreated | | |
| PANCNOT07 | pancreas, fetal, 23 w M | 1 | 0.0142 |
| BRSTUT02 | breast tumor, adenoCA, 54F, | 1 | 0.014 |
| | m/BRSTNOT03 | | |
| TESTTUT02 | testis tumor, embryonal CA, 31M | 1 | 0.0135 |
| PROSNOT16 | prostate, AH, aw/adenoCA, 68M | 1 | 0.0132 |
| LUNGNOT23 | lung, aw/mets osteoSAR, 58M | 1 | 0.0126 |
| BRSTNOT02 | breast, PF changes, aw/adenoCA, | 1 | 0.0111 |
| | 55F, m/BRSTTUT01 | | |

TABLE 6

| | | Tissue | % Abun | Usable in |
|---|---|---|---|---|
| Tissue Category | Found in | Abun | in Found | Found |
| Genitalia, Male | 11/79 | 11 | 0.139 | 41573 |
| Exocrine Glands | 5/43 | 1 | 0.116 | 3889 |
| Pancreas | 2/19 | 2 | 0.105 | 11694 |
| Digestive System | 9/88 | 1 | 0.102 | 3248 |
| Respiratory System | 5/61 | 4 | 0.082 | 25570 |
| Endocrine System | 3/39 | 3 | 0.077 | 10655 |
| Embryonic Structures | 1/15 | 1 | 0.067 | 3472 |
| Nervous System | 6/115 | 1 | 0.052 | 6323 |
| Genitalia, Female | 3/60 | 2 | 0.050 | 7255 |
| Cardiovascular System | 2/59 | 1 | 0.034 | 1346 |
| Urinary Tract | 1/45 | 1 | 0.022 | 3732 |
| Hemic and Immune System | 2/114 | 3 | 0.018 | 7962 |

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. An isolated and purified nucleic acid molecule comprising a nucleic acid sequence encoding a Cid1 polypeptide which has at least 90% identity to the nucleic acid in SEQ ID NO:1, 3, or 5 and complements thereof.

2. The nucleic acid molecule of claim 1 wherein the nucleic acid sequence comprises SEQ ID NO:1.

3. The nucleic acid molecule of claim 1 wherein the nucleic acid sequence comprises SEQ ID NO:3.

4. The nucleic acid molecule of claim 1 wherein the nucleic acid sequence comprises SEQ ID NO:5.

5. The nucleic acid molecule of claim 1 wherein the nucleic acid sequence encodes a polypeptide comprising SEQ ID NO:2

6. The nucleic acid molecule of claim 1 wherein the nucleic acid sequence encodes a polypeptide comprising SEQ ID NO:4.

7. The nucleic acid molecule of claim 1 wherein the nucleic acid sequence encodes a polypeptide comprising SEQ ID NO:6.

8. The nucleic acid molecule of claim 1 which has at least 95% sequence identity to the nucleic acid molecule of claim 2, 3, 4, 5, 6 or 7.

9. The nucleic acid molecule of claim 1 wherein the nucleic acid sequence encodes a polypeptide which has at least 80% sequence identity to the polypeptide encoded by the nucleic acid molecule of claim 5, 6 or 7.

10. The nucleic acid molecule of claim 1 wherein the nucleic acid sequence encodes a polypeptide which has at least 90% sequence identity to the polypeptide encoded by the nucleic acid molecule of claim 5, 6 or 7.

11. The nucleic acid molecule of claim 1 wherein the nucleic acid sequence encodes a polypeptide which has at least 95% sequence identity to the polypeptide encoded by the nucleic acid molecule of claim 5, 6 or 7.

12. An expression cassette comprising the isolated and purified nucleic acid molecule of claim 1 or a biologically active fragment thereof which is operably linked to a promoter functional in a host cell.

13. An isolated and purified polypeptide encoded by the nucleic acid molecule of claim 1.

14. The isolated and purified polypeptide of claim 13 which comprises SEQ ID NO:2.

15. The isolated and purified polypeptide of claim 13 which comprises SEQ ID NO:4.

16. The isolated and purified polypeptide of claim 13 which comprises SEQ ID NO:6.

17. A method of using an isolated nucleic acid molecule, comprising:
transforming host cells with the isolated nucleic acid molecule of claim 1 so as to yield polypeptide encoded by the nucleic acid molecule, or a biologically active portion of the polypeptide.

18. The method of claim 17 further comprising isolating the polypeptide.

19. A method to alter the amount of human polypeptide in a cell, comprising: contacting the cell with the isolated nucleic acid molecule of claim 1 or a biologically active portion thereof operably linked to a promoter so as to alter the amount of the polypeptide in the cell.

20. The method of claim 19 wherein the nucleic acid molecule is in antisense orientation.

21. The method of claim 19 wherein the nucleic acid molecule is in the sense orientation.

22. An isolated polypeptide prepared by the method of claim 18.

23. An isolated antibody specific for the polypeptide of claim 13 or 22.

24. A method to detect a target Cid1 human nucleic acid comprising:
a) contacting a sample with at least one oligonucleotide under conditions effective to amplify said target Cid1 nucleic acid wherein said target Cid1 comprises a nucleic acid selected from the group consisting of SEQ ID NOs:1, 3, 5; and
b) detecting the presence of said target Cid1 nucleic acid.

25. The method of claim 24 wherein two oligonucleotides are employed.

26. The method of claim 24 wherein the amplified nucleic acid comprises at least a portion of SEQ ID NO:1, SEQ ID NO:3, or SEQ ID NO:5.

27. A method to detect the presence of a target nucleic acid comprising:
a) contacting a test sample with a specific polynucleotide wherein said polynucleotide has at least 90% identity to the nucleic acid selected from the group consisting of SEQ ID NOs:1, 3, 5 and complements thereof, and
b) detecting the presence of said target polynucleotide which bind to said specific polynucleotide.

28. The method of claim 27 wherein the human nucleic acid comprises at least a portion of SEQ ID NO:1, SEQ ID NO:3, or SEQ ID NO:5.

29. A method to detect a target Cid1 polypeptide in a test sample Cid1, comprising:
a) contacting said test sample with a specific binding molecule which binds to at least one epitope of an antigen selected from the group consisting of SEQ ID NO 2, 4 and 6, so as to form a complex; and
b) detecting the presence of said complex as an indication of the presence of target Cid1 polypeptide.

30. A method to screen for an agent which inhibits the activity of a Cid1 polypeptide, comprising:
(a) contacting host cells which comprise DNA encoding the Cid1 polypeptide with the agent, wherein said Cid1 polypeptide comprises a polypeptide selected from the group consisting of SEQ ID NOs:2, 4, and 6; and
(b) determining whether the agent inhibits the activity of said Cid1 polypeptide.

31. A method to block Cid 1 expression in a host cell comprising, contacting said host cell with a nucleic acid wherein said nucleic acid down regulates the expression of Cid 1.

32. The method of claim 31 wherein said nucleic acid is selected from the group consisting of antisense molecules, ribozymes or chimeplasty oligonucleotides.

33. The method of claim 29 wherein the host cells are human.

34. A method to identify an agonist of a Cid1 polypeptide, comprising:
a) contacting said Cid1 polypeptide of claim 13 with the agent; and
b) determining whether the agent mimics the activity of said Cid1 polypeptide.

35. An agent identified by the method of claim 31 or 34.


SEQUENCE LISTING (12 sequences)

SEQ ID NO: 1 - DNA, 1397 bp, Homo sapiens
  misc_feature (53)...(53): y = t/u or c at position 53
ctcttattgaagacatggccgccgcgtattttccagactgcatagtcagaccytttggytcctcaggtcaacacttttgggaagttaggatgtgatttggacatgtttttggatctagatgaaaccagaaacctcagcgctcacaagatctcaggaaattttctgatggaatttcaagtgaaaaatgttccttcagaaagaattgcaactcagaagatcctgtctgtgttaggagagtgccttgaccactttggccctggctgtgtgggtgtgcaaaaaatattaaatgcccggtgtccgctcgtgaggttctcacaccaggcctccggatttcagtgtgatttgactacgaacaataggattgccttgacaagttccgaactcctttatatatatggtgccctagactcaagagtgagagccttggtgttcagtgtacggtgctgggctcgagcacattcactaacaagtagtattcctggtgcatggattacaaatttctcccttacaatgatggtcatcttttttctccagagaagatcaccccctattcttccaacactagattccttaaaaaccctagcagatgcagaagataaatgtgtaatagaaggcaacaactgcacatttgttcgtgacttgagtagaattaaaccttcacagaacacagaaacattagaattactactgaaggaattttttgagtattttggcaattttgctttcgataaaaattccataaatattcgacagggaagggagcaaaacaaacctgattcttctcctctgtacattcagaatccatttgaaacttctctcaacataagcaaaaatgtaagtcaaagccagctgcaaaaatttgtagatttggcccgagaaagtgcctggattttacaacaggaagatacagatcgaccttccatatcaagtaatcggccctgggggctggtatccctattgctaccatctgctccaaacagaaagtcctttaccaagaagaaaagcaataagtttgcaattgaaacagtcaaaaacttgctagaatctttaaaaggtaacagaacagaaaatttcacaaaaaccagtgggaagagaacaattagtactcagacatgatggctgctacattgtgtaaagaactgggcttagcctatcaaatggtctgtggacttacttggaaaaactgatttgaaactttcacagatctcagctttcatctgatgtcacttttcatgatcttctcattggcccccttaacctggtctgaagttctgggatgttttcagtttgatcagtctgatactcagtggcactttattaaaacatcagctgtggagtgtggcggtgcacacctgtagtcccagctgctcaggaggctnaggaagaggantnntnag

SEQ ID NO: 2 - protein, 337 aa, Homo sapiens [sequence omitted]

SEQ ID NO: 3 - DNA, 1543 bp, Homo sapiens
  misc_feature (2)...(3): n = a or g or c or t/u, unknown, or other at positions 2 and 3
annaagatcaggctcgtgaacatattcggcaaaacctagaaagtttcataagacaggactttccaggaactaaattgagcctgtttggctcctccaaaaatggatttgggttcaaacagagtgaccttgacgtctgtatgacaattaatggacttgaaactgctgagggattggactgtgtcagaactattgaagaattagcaagagtcctcagaaaacattcaggtctgagaaacatcttacctattacaacagcaaaggtgccaattgtgaagttcttccatttgagaagtggtctggaagtagatatcagtttgtataacacattggcccttcataacacaaggcttttatctgcttattccgccattgatcccagagtgaagtatttgtgctataccatgaaagtatttacaaagatgtgtgatattggtgatgcatctagaggcagcttatcatcgtatgcatatactcttatggtgctatattttctccagcagaggaatccaccagtcattcctgtccttcaagagatatacaaaggtgaaaagaaacctgaaatatttgttgatggctggaatatttatttttttgatcaaatagatgaactgcctacctattggtcagaatgtggaaaaaatacagaatctgttgggcagttatggttgggccttcttcgtttctacacagaggaatttgattttaaagaacatgttattagcatcaggagaaaaagtctgcttacaacttttaagaaacagtggacctcaaaatacattgttattgaagatccctttgatttgaatcataatcttggagctggattatcaaggaaaatgacaaattttataatgaaggcttttatcaatggwagaagagtatttggtattcctgtcaagggatttccaaaggactacccctcaaaaatggaatacttttttgatccagatgtgttaactgaaggagagctggccccaaatgatagatgttgtcgaatttgtggaaaaatcggacacttcatgaaggactgtcctatgaggagaaaagtaagacggcggcgagatcaggaagatgccctgaaccaaagataccctgagaacaaggaaaaaagaagcaaagaggacaaagaaattcacaacaagtacacagaaagggaggtgtcaacaaaagaagataagcccatacagtgcacacctcagaaagccaagccaatgcgggcagctgctgacctggggagggagaagatcctcaggccaccagtagaaaaatggaagagacaggatgacaaagacttaagagaaaaacgttgttttatttgtggaagagaagggcacattaaaaaggaatgcccacagtttaaaggctcttcaggtagcctttccagtaaatatatgactcagggaaaagcctcagcgaagaggacccagcaggaatcatgagggaaggaaaatgcagcactctaaatggccactcaggcgttcctattcactcggaaaattaggttcatttcacaggacacagcagtgtagatcaggcttcaact

SEQ ID NO: 4 - protein, 433 aa, Homo sapiens
MTINGLETAEGLDCVRTIEELARVLRKHSGLRNILPITTAKVPIVKFFHLRSGLEVDISLYNTLALHNTRLLSAYSAIDPRVKYLCYTMKVFTKMCDIGDASRGSLSSYAYTLMVLYFLQQRNPPVIPVLQEIYKGEKKPEIFVDGWNIYFFDQIDELPTYWSECGKNTESVGQLWLGLLRFYTEEFDFKEHVISIRRKSLLTTFKKQWTSKYIVIEDPFDLNHNLGAGLSRKMTNFIMKAFINGRRVFGIPVKGFPKDYPSKMEYFFDPDVLTEGELAPNDRCCRICGKIGHFMKDCPMRRKVRRRRDQEDALNQRYPENKEKRSKEDKEIHNKYTEREVSTKEDKPIQCTPQKAKPMRAAADLGREKILRPPVEKWKRQDDKDLREKRCFICGREGHIKKECPQFKGSSGSLSSKYMTQGKASAKRTQQES

SEQ ID NO: 5 - DNA, 2138 bp, Homo sapiens
  misc_feature (2)...(2): n = a or g or c or t/u, unkown or other at position 2
gnggcagcttcgcagcctagtggtggccctgatgcaggangtcttcacagagttcttccctggctgtgtggtccacccttttggctcttccataaatagcttcgatgtccatggctgtgatcttgacctcttcttggatctgggtgacttggaagagccccagccagtcccaaaggctccagaatctccatcgctggactcggccctgggttccccactggaccctcaagccctggcctgcaccccagcttcccctccagattcacaacctcctgcttctccccaggattctgaagccctggactttgaaaccccttcctcctccctggcgccccaaactccggactctgccttggsctccgagacccttgcttctccccagtctctgcctscagcttcaccactgctagaggacagggaagagggggacctggggaaggyctcggaactagcagagaccccaaaggaggagaaagcagaggrgscagcaatgctggagctggtgggatccattctccggggctgtgtccctggggtgtatcgagtccaaactgtgccctstgcccggcgmcctgtggtcaagttctgtcatcggccttcaggtctccacggtgatgtctccctcagtaaccggctggccctgcataactcccgtttcctgagtctctgctctgagctggatggtcgagtccggcccctcgtgtacaccctccgctgctgggctcagggtcgggggctgtcagggagtggcccccttctcagtaactacgccctgaccttgctggtgatctattttcttcagaccagggaccctcctgtgttgcccactgtgtcccagctcacccagaaagcaggagagggggaacaggtggaagtcgatggctgggactgcagtttccccagggatgcctcaagactggagcccagcataaatgtggagcccctcagttccctgctagcccagttcttctcctgtgtatcttgttgggatcttcgtggctccctgctgtccctgcgggagggtcaggcactgcctgtggcagggggcctgccttctaatctctgggagggtctgcgccttggccccctgaatctccaggacccttttgacctgagtcacaatgtcgcagccaatgtgaccagccgggtggctgggcgcctacagaactgctgccgagcagcagccaattactgccgaagcctccagtaccagcgccgttcctcccggggtcgggactgggggctgctccctcttctgcagcccagctcccccagctccctgctctctgctacgccgatccctttaccccttgcacccttcacccagctcactgctgccctggtgcaggtattcagggaagcactggggtgccatatagaacaggcaaccaagagaacgcggtcagaaggaggtggaactggggagtcctctcagggagggacaagcaaaagactcaaagtagatggacagaaaaactgctgtgaggaggggaaagaggagcagcagggatgtgcaggggacggtggggaagacagggtagaagagatggttatagaggttggagagatggtgcaggactgggccatgcagagccctgggcagccaggggacctgcccctgaccactggaaagcatggagcccctggagaagaggggcagcccagccacgcagccctggcagagcgggggcccaagggacatgaggcagcccaagaatggtctcagggtgaggcagggaagggggcatccctgccctcctcagcgagctggcgctgtgccttgtggcaccgagtgtggcaagggcggcggcgagcccgtagacgcttgcagcagcaaaccaaggagggagctggaggtggcgctggcacaagagcagggtggctggcgactgaggctcaggtcacccaggagctgaaaggactgagtggtggcgaagagaggccagaaactgagcccctgctgagctttgtggcgtctgtctccccggctgaccgaatgctcactgtgaccccgctccaggatccccaaggcctgttccctgatctccatcatttcttacaggttttcctccctcaagcaattcgacatctcaagtgaagacatggcccctgaagggcaataaagctgctagtttatt

SEQ ID NO: 6 - protein, 688 aa, Homo sapiens
  VARIANT (3)...(3): Xaa = Unknown or other at position 3
MQXVFTEFFPGCVVHPFGSSINSFDVHGCDLDLFLDLGDLEEPQPVPKAPESPSLDSALGSPLDPQALACTPASPPDSQPPASPQDSEALDFETPSSSLAPQTPDSALXSETLASPQSLPXASPLLEDREEGDLGKXSELAETPKEEKAEXXAMLELVGSILRGCVPGVYRVQTVPXARRPVVKFCHRPSGLHGDVSLSNRLALHNSRFLSLCSELDGRVRPLVYTLRCWAQGRGLSGSGPLLSNYALTLLVIYFLQTRDPPVLPTVSQLTQKAGEGEQVEVDGWDCSFPRDASRLEPSINVEPLSSLLAQFFSCVSCWDLRGSLLSLREGQALPVAGGLPSNLWEGLRLGPLNLQDPFDLSHNVAANVTSRVAGRLQNCCRAAANYCRSLQYQRRSSRGRDWGLLPLLQPSSPSSLLSATPIPLPLAPFTQLTAALVQVFREALGCHIEQATKRTRSEGGGTGESSQGGTSKRLKVDGQKNCCEEGKEEQQGCAGDGGEDRVEEMVIEVGEMVQDWAMQSPGQPGDLPLTTGKHGAPGEEGQPSHAALAERGPKGHEAAQEWSQGEAGKGASLPSSASWRCALWHRVWQGRRRARRRLQQQTKEGAGGGAGTRAGWLATEAQVTQELKGLSGGEERPETEPLLSFVASVSPADRMLTVTPLQDPQGLFPDLHHFLQVFLPQAIRHLK

SEQ ID NO: 7 - protein, 355 aa, Schizosaccharomyces pombe
VYNEIKISDKEFKEKRAALDTLRLCLKRISPDAELVAFGSLESGLALKNSDMDLCVLMDSRVQSDTIALQFYEELIAEGFEGKFLQRARIPIIKLTSDTKNGFGASFQCDIGFNNRLAIHNTLLLSSYTKLDARLKPMVLLVKHWAKRKQINSPYFGTLSSYGYVLMVLYYLIHVIKPPVFPNLLLSPLKQEKIVDGFDVGFDDKLEDIPPSQNYSSLGSLLHGFFRFYAYKFEPREKVVTFRRPDGYLTKQEKGWTSATEHTGSADQIIKDRYILAIEDPFEISHNVGRTVSSSGLYRIRGEFMAASRLLNSRSYPIPYDSLFEEAPIPPRRQKKTDEQSNKKLLNETDGDNSE

SEQ ID NO: 8 - protein, 305 aa, Schizosaccharomyces pombe
DMDLCVLMDSRVQSDTIALQFYEELIAEGFEGKFLQRARIPIIKLTSDTKNGFGASFQCDIGFNNRLAIHNTLLLSSYTKLDARLKPMVLLVKHWAKRKQINSPYFGTLSSYGYVLMVLYYLIHVIKPPVFPNLLLSPLKQEKIVDGFDVGFDDKLEDIPPSQNYSSLGSLLHGFFRFYAYKFEPREKVVTFRRPDGYLTKQEKGWTSATEHTGSADQIIKDRYILAIEDPFEISHNVGRTVSSSGLYRIRGEFMAASRLLNSRSYPIPYDSLFEEAPIPPRRQKKTDEQSNKKLLNETDGDNSE

SEQ ID NO: 9 - protein, 405 aa, Schizosaccharomyces pombe
MNISSAQFIPGVHTVEEIEAEIHKNLHISKSCSYQKVPNSHKEFTKFCYEVYNEIKISDKEFKEKRAALDTLRLCLKRISPDAELVAFGSLESGLALKNSDMDLCVLMDSRVQSDTIALQFYEELIAEGFEGKFLQRARIPIIKLTSDTKNGFGASFQCDIGFNNRLAIHNTLLLSSYTKLDARLKPMVLLVKHWAKRKQINSPYFGTLSSYGYVLMVLYYLIHVIKPPVFPNLLLSPLKQEKIVDGFDVGFDDKLEDIPPSQNYSSLGSLLHGFFRFYAYKFEPREKVVTFRRPDGYLTKQEKGWTSATEHTGSADQIIKDRYILAIEDPFEISHNVGRTVSSSGLYRIRGEFMAASRLLNSRSYPIPYDSLFEEAPIPPRRQKKTDEQSNKKLLNETDGDNSE

SEQ ID NO: 10 - DNA, 1623 bp, Schizosaccharomyces pombe
tgtaactgacttgcttcaattagtaaaaggaagcatcaaagaccattaaacagtgaataattactcaaagagaaagtgaataagtttgccactttcaccttttgagcagtagacgaatatgaacatttcttctgcacaatttattcctggtgttcacacagttgaagagattgaggcagaaattcacaaaaatttacatatttcaaaaagttgtagctaccaaaaggtccctaattcgcacaaggaatttacgaagttttgctatgaagtgtataatgagattaaaattagtgacaaagagtttaaagaaaagagagcggcattagatacacttcggctatgccttaaacgaatatcccctgatgctgaattggtagcctttggaagtttggaatctggtttagcacttaaaaattcggatatggatttgtgcgtgcttatggattcgcgcgtccaaagtgatacaattgcgctccaattctatgaagagcttatagctgaaggatttgaaggaaaatttttacaaagggcaagaattcccattatcaaattaacatctgatacgaaaaatggatttggggcttcgtttcaatgtgatattggatttaacaatcgtctagctattcataatacgcttttactttcttcatatacaaaattagatgctcgcctaaaacccatggtccttcttgttaagcattgggccaaacggaagcaaatcaactctccttactttggaactctttccagttatggttacgtcctaatggttctttactatctgattcacgttatcaagcctcccgtctttcctaatttactgttgtcacctttgaaacaagaaaagatagttgatggatttgacgttggttttgacgataaactggaagatatccctccttcccaaaattatagctcattgggaagtttacttcatggcttttttagattttatgcttataagttcgagccacgggaaaaggtagtaacttttcgtagaccagacggttacctcacaaagcaagagaaaggatggacttcagctactgaacacactggatcggctgatcaaattataaaagacaggtatattcttgcgattgaagatcctttcgagatttcacataatgtgggtaggacagttagcagttctggattgtatcggattcgaggggaatttatggccgcttcaaggttgctcaattctcgctcatatcctatcccttatgattcattatttgaggaggccccaattccgcctcgtcgccagaaaaaaacggatgaacaatctaacaaaaaattgttgaatgaaaccgatggtgacaattctgagtgattttacaaagaagtcttaaaaagcgcatcaatattcattcccctagattcatagtaatcattcattattactctgcttttatttgcttacttttgaaagcatggaatgtttttatttttctttaaaaattttgacctcttgtttctaatacctttccccctttgttttatgtacaacctcttgttaatagtctacttgttggtgagaaaataggctcatcatagttaacatcaatgtccaaaaaattttcatagatctataattaataattatttattgcaaatgct

SEQ ID NO: 11 - protein, 313 aa, Schizosaccharomyces pombe
MTINGLETAEGLDCVRTIEELARVLRKHSGLRNILPITTAKVPIVKFFHLRSGLEVDISLYNTLALHNTRLLSAYSAIDPRVKYLCYTMKVFTKMCDIGDASRGSLSSYAYTLMVLYFLQQRNPPVIPVLQEIYKGEKKPEIFVDGWNIYFFDQIDELPTYWSECGKNTESVGQLWLGLLRFYTEEFDFKEHVISIRRKSLLTTFKKQWTSKYIVIEDPFDLNHNLGAGLSRKMTNFIMKAFINGRRVFGIPVKGFPKDYPSKMEYFFDPDVLTEGELAPNDRCCRICGKIGHFMKDCPMRRKVRRRRDQEDA

SEQ ID NO: 12 - protein, 477 aa, Schizosaccharomyces pombe
  VARIANT (3)...(3): Xaa = Unknown or other at position 3
MQXVFTEFFPGCVVHPFGSSINSFDVHGCDLDLFLDLGDLEEPQPVPKAPESPSLDSALGSPLDPQALACTPASPPDSQPPASPQDSEALDFETPSSSLAPQTPDSALXSETLASPQSLPXASPLLEDREEGDLGKXSELAETPKEEKAEXXAMLELVGSILRGCVPGVYRVQTVPXARRPVVKFCHRPSGLHGDVSLSNRLALHNSRFLSLCSELDGRVRPLVYTLRCWAQGRGLSGSGPLLSNYALTLLVIYFLQTRDPPVLPTVSQLTQKAGEGEQVEVDGWDCSFPRDASRLEPSINVEPLSSLLAQFFSCVSCWDLRGSLLSLREGQALPVAGGLPSNLWEGLRLGPLNLQDPFDLSHNVAANVTSRVAGRLQNCCRAAANYCRSLQYQRRSSRGRDWGLLPLLQPSSPSSLLSATPIPLPLAPFTQLTAALVQVFREALGCHIEQATKRTRSEGGGTGESSQGGTSKRLKV